(19) United States
(12) Patent Application Publication
(10) Pub. No.: US 2025/0177988 A1
(43) Pub. Date: Jun. 5, 2025

(54) DETERMINING A TEMPERATURE OF A FIRST SHEET OF A WALL OF A POLYMERASE CHAIN REACTION (PCR) WELL BASED ON A RESISTANCE AND A CORRECTION MODEL

(71) Applicant: Hewlett-Packard Development Company, L.P., Spring, TX (US)

(72) Inventors: Napoleon J. LEONI, Palo Alto, CA (US); Omer GILA, Palo Alto, CA (US)

(73) Assignee: Hewlett-Packard Development Company, L.P., Spring, TX (US)

(21) Appl. No.: 18/844,032

(22) PCT Filed: Mar. 24, 2022
(86) PCT No.: PCT/US2022/021803

§ 371 Date: Sep. 4, 2024

(51) Int. Cl. B01L 7/00; B01L 3/00; C12Q 1/6844

(52) U.S. Cl. CPC B01L 7/52; B01L 3/502715; C12Q 1/6844; B01L 2300/0663; B01L 2300/1827

(57) ABSTRACT

A device includes a well and a control portion. The well is to receive a polymerase chain reaction (PCR) mixture within an interior partially defined by a first wall, which comprise a heating first sheet. The control portion is to determine a first temperature of a first portion of the first sheet exposed to the interior, based on monitoring a resistance of the first sheet and based on a correction model, the correction model representing a thermal behavior of at least one structure which further defines the well and which is in thermal relation with at least a second portion the first sheet. The control portion is also, based on the determined first temperature, to apply a first electrical signal to the heating first sheet to heat the PCR mixture, at a selectable temperature, within a thermal cycling zone within the interior in close thermal proximity to the first wall.

### BACKGROUND

Molecular diagnostics has revolutionized modern medicine. For example, molecular diagnostics have been used to better detect infectious diseases, obtain genetic information, perform pharmacogenomics, facilitate oncology, and for other purposes. Some types of molecular diagnostics may employ polymerase chain reaction (PCR) processes to rapidly make many copies of nucleic acid strands, such as RNA and/or DNA strands.

### BRIEF DESCRIPTION OF THE DRAWINGS

FIG. 1A is a diagram including a sectional side view schematically representing an example testing device (and/or example testing method) including a well to perform polymerase chain reaction (PCR) testing.

FIGS. 1B and 1D each are a diagram including a sectional side view schematically representing an example testing device (and/or example testing method) for sensing a temperature of a first wall of a PCR well.

FIG. 1C is a graph schematically representing an example relationship between resistance of a first sheet of a PCR well and temperature.

FIGS. 2 and 3 are each a diagram including a sectional side view schematically representing an example testing device (and/or example testing method) including an example well to receive a polymerase chain reaction (PCR) mixture.

FIG. 4 is a diagram including a sectional side view schematically representing an example testing device including a plurality of example wells, each to receive a polymerase chain reaction (PCR) mixture.

FIG. 5 is a diagram including a sectional side view schematically representing an example testing device including a PCR well to receive releasable engagement from a heat sink.

FIG. 6 is a diagram including a sectional side view schematically representing an example testing device including a PCR well and heat sink releasably engaged relative to each other, and a heat sink control system associated with the heating block.

FIG. 7 is a block diagram schematically representing an example cooling element including different example cooling modalities.

FIG. 8 is a block diagram schematically representing example signal source modalities of an example device and/or example method.

FIG. 9A is a graph schematically representing an example temperature profile during an example method of thermal cycling a PCR mixture.

FIG. 9B is a graph schematically representing example power application profile for a heating sheet of a PCR well during an example method of thermal cycling of a PCR mixture.

FIG. 9C is a graph schematically representing example power application profile for numerically simulating a comparison of sensed temperature heat sink during an example method of thermal cycling of a PCR mixture.

FIG. 10A is diagram including a sectional side view schematically representing an example testing device (and/or example testing method) including an example well to receive a polymerase chain reaction (PCR) mixture.

FIG. 10B is a graph schematically representing a comparison of example temperature profiles based on measuring a resistance of a heating element of an example well for receiving a polymerase chain reaction (PCR) mixture.

FIG. 10C is a graph schematically representing an example test variable power signal input profiles.

FIG. 11A is a diagram schematically representing an example device, including circuitry, to apply a correction model when applying heat at selectable temperatures via a heating element of an example PCR well.

FIG. 11B is a diagram schematically representing an example filter used in association with one example correction model.

FIG. 11C is a diagram schematically representing an example portion for selecting and/or adjusting filter parameters for a correction model.

FIG. 12 is a block diagram schematically representing an example operations engine.

FIGS. 13A and 13B are each a block diagram schematically representing an example control portion and an example user interface, respectively.

FIG. 14 is a flow diagram of an example method including performing a polymer chain reaction (PCR) test via an example PCR well.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

Among other purposes, molecular diagnostics may help identify infectious diseases. One class of molecular diagnostics includes a nucleic acid amplification test, such as a polymerase chain reaction (PCR) assay to amplify target genomic material for detection. One common use of such PCR testing is for detecting viral genomic material such as, but not limited to, detecting a virus like Severe Acute Respiratory Syndrome Corona Virus 2 (SARS-CoV2), which may sometimes be referred to as COVID-19 (i.e. Corona Virus Disease of 2019).

In general terms, such nucleic acid amplification is used to make many copies of a genetic sample in order to greatly increase the accuracy and reliability of detection of the analyte of interest, such as viral genetic material. One measure of the effectiveness of such nucleic acid amplification may comprise a limit of detection, which may informally be referred to as the lowest concentration of an analyte that can be reliably detected. In general terms, the lower the limit of detection, the more accurate and reliable the test such that fewer false negatives are reported.

In general terms, the PCR assay is carried out by isolating nucleic acid strands, such as DNA strands or RNA strands, from a sample from biological material and adding it to a PCR master mix in a well of a testing device to form a PCR mixture. Biological materials may comprise human, animal, microbial, or plant biological material. In some examples, the biological material may be obtained from a human patient sample.

In most instances, because of complexity, cost, and other factors, the testing device may be located in a laboratory. However, some testing devices may be mobile and deployable in the field, such as at a point-of-care. In view of the considerable societal impact caused by some infectious diseases, providing faster and/or more mobile testing may ease the impact of such diseases by enabling early detection, tracking patterns of disease migration, treatment effectiveness, and public health decision-making.

With further reference to the actual testing, in some examples a PCR master mix, to which a sample of biological material (e.g. biological sample) is added to form a PCR mixture, may comprise components to execute the basic steps of a polymerase chain reaction via thermal cycling within an appropriately sized test well. This combination of the PCR master mix and the genetic sample may sometimes be referred to as a PCR mixture or a PCR sample volume. In some instances, a PCR sample volume may comprise about 25 to about 50 microliters and a testing device may provide for testing a group of wells arranged as a well plate or well chip. Via the testing device, a combination of the biological sample and the PCR master mix comprises the PCR sample volume, which is heated within each test well in a manner to perform the PCR assay.

Among other components, a PCR mixture may comprise a template nucleic acid sequence (e.g. DNA strands, RNA strands, portions thereof) and a PCR master mix, which may comprise buffers, dyes, cofactors, beads, primers (e.g. forward primer, reverse primer), probes, deoxyribose nucleotides (dNTPs), and/or enzyme DNA polymerase. The template nucleic acid sequence corresponds to the known target nucleic acid sequence to be amplified. In some examples, the enzyme DNA polymerase may comprise a Taq DNA polymerase such as a thermophilic eubacterial microorganism (Thermus aquaticus). In some examples, the cofactor may comprise Magnesium Chloride or Magnesium Sulfate. In some examples, the water may comprise nuclease-free water or PCR-grade water. In some instances, the PCR master mix also may be referred to as a PCR super mix or a PCR ready mix. Once the components appropriate for a desired type of PCR test are selected, they are added at appropriate concentrations in combination to prepare a batch mixture, which is then divided among multiple PCR wells. A volume of a PCR mixture (i.e. PCR sample volume), which includes the genetic sample and the PCR master mix per well may comprise 25 to 50 microliters (μL). At least some example PCR master mixes are available commercially from a number of sources such as, but not limited to, Sigma-Aldrich, Inc. of Saint Louis, Missouri, United States or at www.sigmaaldrich.com.

A PCR assay test involves thermal cycling, which may comprise a first “denaturation” step (i.e. phase) in which the PCR sample volume (i.e. PCR mixture) is heated to a temperature of at least about 90 degrees Celsius up to about 98 degrees Celsius (or slightly higher temperatures), which causes double-stranded nucleic acid (DNA or RNA) within the PCR mixture (i.e. PCR sample volume) to melt apart by breaking the hydrogen bonds between complementary bases, yielding two single-stranded nucleic molecules. In some examples, denaturation temperature might exceed 100 degree Celsius for a short period of time for up to a few milliseconds, in some examples. A second step (i.e. phase) in the thermal cycling of a PCR assay test may comprise annealing in which less heat is applied to lower the reaction temperature to about 50-65° C., which allows annealing of the primers to each of the single-stranded nucleic acid templates as part of the reaction process. A third step (i.e. phase) of the thermal cycling of a PCR assay test may comprise elongation (i.e. extension) in which the heat applied to the PCR sample volume is selected to create a reaction temperature suitable for the particular nucleic acid (e.g. DNA) polymerase used. In some examples, one target activity temperature for a thermostable nucleic acid polymerase including Taq polymerase (e.g. a thermophilic eubacterial microorganism, Thermus aquaticus) is approximately 65-80° C. In this third step, the nucleic acid polymerase synthesizes a new nucleic acid strand complementary to the nucleic acid template strand by adding free nucleotide triphosphates (dNTPs) from the reaction mixture. In some examples, the different temperatures used in these three different temperature phases of thermal cycling may vary depending on the length of the nucleic acid strand, the time available, the type of target (e.g. RNA, DNA, etc.), the density of polymerase and primers, and/or other parameters.

Some types of implementing a PCR assay test may include combining the second and third phases (annealing and elongation) by heating the PCR sample mixture (after a denaturation phase) within a temperature range having values somewhere between the above-mentioned respective annealing and elongation temperature ranges to contemporaneously perform annealing and elongation.

In general terms, repetition of thermal cycles in performing the PCR assay test may result in an exponential increase in the quantity of the target nucleic acid sequence (e.g. DNA or RNA) to be amplified, which may sometimes be referred to the amplicon. Each cycle doubles the number of nucleic acid molecules (amplicons) amplified from the nucleic acid sequence template. For instance, in some implementations, repeating the PCR process for 30 cycles may produce on the order of 2³⁰ molecules of the target nucleic acid sequence. Of course, the number of cycles may vary depending on amplification efficiency, detection limit, or the analyte of interest, with some PCR processes utilizing thermal cycles between about 20 to about 40 cycles.

Once a sufficient number of cycles has been performed to obtain a desired quantity of the amplicons, the testing device hosting the PCR well(s) (or another testing device) may be used to detect the analyte of interest.

In some arrangements, optical detection may be used to detect output elements of the PCR assay test. This optical detection may be expressed as output element signal intensity, which may indicate a presence, a quantity, and/or a concentration of a particular analyte (e.g. virus particle, other) to which the output element is attached (e.g. bonded). In some arrangements, detection of the output elements also may be used to determine the progression of a PCR assay test.

In some arrangements, such optically-detectable output elements may comprise a fluorescent agent (e.g. dye), which may form part of the PCR master mix. One fluorescent agent may comprise a fluorophore which comprises a fluorescent chemical compound that can re-emit light upon light excitation. With this in mind, a PCR test well may comprise a wall or cover which permits the transmission of light into and/or through the PCR mixture within the test well to enable optical detection of such fluorophores to determine a presence, quantity, and/or concentration of a particular analyte.

It will be understood that other methods may be used to detect a presence, quantity, and/or concentration of the target nucleic acid (e.g. DNA or RNA) sequence of interest after amplification by the PCR process.

With this general context in mind, in at least some examples of the present disclosure, an example method and/or example device is directed to increasing accuracy in sensing a temperature of at least a portion of PCR mixture during selective local heating of the PCR mixture in different temperature phases during a PCR test. In some examples, the PCR test well and PCR mixture provide for a pulse-controlled amplification type of PCR test, as further described later.

In some examples of the present disclosure, a device comprises a well and a control portion. The well is to receive a polymerase chain reaction (PCR) mixture within an interior partially defined by a first wall, which comprises a heating first sheet. The control portion is to determine a first temperature of a first portion of the first sheet exposed to the interior, based on monitoring a resistance of the first sheet and based on a correction model, the correction model representing a thermal behavior of at least one structure which further defines the well and which is in thermal relation with at least a second portion of the first sheet. Based on the determined first temperature, a first electrical signal is applied to the heating first sheet to heat the PCR mixture, at a selectable temperature, within a thermal cycling zone within the interior in close thermal proximity to the first wall.

In some examples, an example device and/or example method may comprise sensing a temperature of a first sheet of the first wall of the PCR well. In some such examples, sensing a temperature of the first sheet may be based on a resistance of the first sheet, such as changes in resistance. Because the first sheet is directly exposed to, and in direct thermal relation with, a portion of the PCR mixture in the PCR well which is within a thermal cycling zone (e.g. in close thermal proximity to the first sheet), a temperature of the portion of the PCR mixture may directly correspond to the temperature of the first sheet, subject to application of the previously-mentioned example correction model. Accordingly, in general terms, a determination of changes in resistance of the first sheet throughout the course of a PCR test, such as during the different temperature phases, provides a corresponding indication of the temperature of the first sheet, and therefore an indication of the temperature of the PCR mixture, at least with regard to a portion of the PCR mixture within the thermal cycling zone. In some such examples, a generally linear relationship exists between the changes in resistance (R/R₀) of the first sheet and the temperature the first sheet.

In some examples, the control portion is to determine the first temperature of the first sheet based on a temperature coefficient of resistivity of the first sheet and a linear relationship of the determined first temperature of the first sheet and the resistance. In some examples, the temperature coefficient of resistivity may be pre-determined.

In some examples, the monitored resistance of the first sheet corresponds to an average temperature of the first sheet. In some such examples, this average temperature refers to a temperature of the entire first sheet, which includes both of the first portion of the first sheet exposed to the PCR mixture and the second portion of the first sheet which is not exposed to the PCR mixture.

In some examples, the control portion is to determine the first temperature of the first portion of the first sheet, by generating via the correction model, a temperature correction value to be applied to the average temperature of the first sheet, wherein the correction model at least partially represents the thermal behavior according to at least one of: a heating power of the first sheet during heating; and a cooling power associated with the at least one structure, which comprises a heat sink in indirect thermal contact with the first sheet.

In some examples, the correction model is to at least partially represent the thermal behavior by digitally filtering the heating power to remove at least one of: a first temperature change occurring slower than an overall thermal response of the well; and a second temperature change faster than local time constants of the first sheet.

In some examples, the correction model is to at least partially represent the thermal behavior by digitally filtering the cooling power to remove at least one of: a third temperature change occurring slower than the overall thermal response of the well connected to a heat sink; and a fourth temperature change faster than local time constants of the well.

In some examples, the thermal behavior represented by the correction model comprises a temperature coefficient of resistivity and model filter parameters, each of which are based on at least one of: a geometry of at least the first sheet; a type of material of the first sheet; a sensitivity of the resistance of the first sheet to temperature changes; and a geometry of the at least one structure further defining the well.

In some examples, the at least one structure further defining the well comprises at least second walls defining, in combination with the first wall, the interior of the well.

In some examples, the device comprises a control portion to cause, via an electrical signal, the first sheet to generate heat in a thermal cycling zone within the interior in close thermal proximity to the first wall in different phases operating in sequence at: a first temperature comprising at least about 90 degrees Celsius (° C.); a second temperature comprising at least about 25° C. less than the first temperature; and a third temperature comprising at least about 5° C. greater than the second temperature and at least about 15° C. less than the first temperature.

In some examples, the control portion is to cause operation of the different heating phases as: a first phase operating at the first temperature and comprising a first duration comprising no more than 10 milliseconds; a second phase operating at the second temperature and comprising a duration of at least 300 milliseconds; and a third phase operating at the third temperature and comprising a duration of at least 300 milliseconds.

In some examples, the different temperature phases by which a PCT test is performed via the example device may comprise a first phase operating at the first temperature for a first duration of no more than about 10 milliseconds (e.g. 9.5, 9.6, 9.7, 9.8, 9.9, 10, 10.1, 10.2, 10.3, 10.4, 10.5 milliseconds), a second phase operating at the second temperature for a second duration of at least about 300 milliseconds (e.g. 298, 299, 300, 301, 302 milliseconds), and a third phase operating at the third temperature for a third duration of at least about 300 milliseconds (e.g. 298, 299, 300, 301, 302) milliseconds). In some examples, the second phase may operate at the second temperature for a second duration of at least about 400 milliseconds (e.g. 398, 399, 400, 401, 402 milliseconds), and a third phase may operate at the third temperature for a third duration of at least about 400 milliseconds (e.g. 398, 399, 400, 401, 402 milliseconds). In some examples, the second phase may operate at the second temperature for a second duration of at least about 500 milliseconds (e.g. 498, 499, 500, 501, 502 milliseconds), and a third phase may operate at the third temperature for a third duration of at least about 500 milliseconds (e.g. 498, 499, 500, 501, 502 milliseconds).

Via such arrangements, in some examples, a total duration of a thermal cycle may comprise less than about 1.5 (e.g. 1.45, 1.46, 1.47, 1.48, 1.49, 1.5, 1.51, 1.52, 1.53, 1.54, 1.55) seconds. Other examples are further described below.

In one aspect, at least some examples of the present disclosure provide for closed loop control over temperature of the first sheet, and therefore of the portion of the PCR mixture within the thermal cycling zone.

This arrangement stands in sharp contrast to the non-example arrangements in which an average temperature of the heating first sheet was used to perform the closed loop control, and in which a temperature of the PCR mixture (at least the portion within a thermal cycling zone) may not be accurately represented.

These challenges posed by such non-example arrangements may then affect the accuracy and/or sensitivity of testing performed via such PCR wells because the desired target temperatures of the PCR mixture (or portions thereof) cannot be achieved at all and/or at the right time, thereby decreasing the effectiveness and/or efficiency of the PCR process.

Accordingly, if one were to attempt using an average temperature of a heating first sheet of a PCR well to perform closed loop controlled heating as in some non-example arrangements, such non-example arrangements may result in a decrease in the degree of amplification during each thermal cycle, thereby reducing the total quantity of amplified genetic sample (e.g. amplicon). This diminished quantity of the amplicon, in turn, may increase the limit of detection, thereby potentially reducing the accuracy and/or reliability of the particular PCR test, which may thereby increase a number of false negatives.

Alternatively, if more time were provided to wait for such non-example arrangements to complete a desired amount of annealing and/or elongation, then the duration of each thermal cycle would be significantly longer, and then an overall duration of the complete PCR test would become significantly longer, which would be counter-productive for achieving more rapid, highly accurate PCR testing.

In sharp contrast, at least some examples of the present disclosure may provide for significantly increased accuracy in temperature control of a PCR mixture at least by applying a correction model to a determined temperature based on sensing a resistance of the heating first sheet.

Via such examples of the present disclosure, upon performing a PCR test in which annealing and elongation occur in separate phases at different target temperatures, and such temperatures are achieved via local heating by the first sheet (e.g. heating wall) of the PCR well (which is directly exposed to the PCR mixture), the arrangement of increased accuracy in sensing a temperature of the PCR mixture (at least the portion within the thermal cycling zone) may enhance the amount of annealing and/or elongation which may occur within a given duration and/or may enhance accurate, timely transitions between an annealing temperature and an elongation temperature.

This example arrangement, in turn, results in a desired degree of amplification during each cycle, thereby producing an acceptable total quantity of amplified genetic sample (e.g. amplicon). Reliably and quickly achieving a desired total quantity of the amplicon may, in turn, enhance a limit of detection for the particular analyte of interest, which may increase the accuracy and/or reliability of the particular PCR test, such as via decreasing the number of false negatives.

As previously mentioned, in some examples of the present disclosure, the PCR test may comprise a pulse-controlled amplification (PCA) type of PCR test. In a pulse-controlled amplification type of PCR test, instead of subjecting the entire volume of the PCR mixture (i.e. PCR sample volume) in the PCR well to thermal cycling, a relatively small percentage of the PCR sample volume is subjected to the thermal cycling at any given time. In some such examples of pulse-controlled amplification, heat is applied locally in a thermal cycling zone within an interior of the PCR well in close thermal proximity to a wall (e.g. second wall) of the PCR well such that just the portion of the PCR sample volume within the thermal cycling zone is subjected to the respective target temperatures of each of the three different temperature phases, as previously described. In some such examples, the local heating comprises applying heat in a rapid initial pulse (e.g. on the order of microseconds) in order to achieve the first temperature of the first phase of the PCR test, with each pulse followed by a relatively rapid cooling upon the cessation of the initial heating pulse, which is then followed (after a short pause in some examples) in further heating, at selectable power levels, via the first sheet to maintain for a selectable duration, a second temperature, and then to maintain for a selectable duration, a third temperature to complete a thermal cycle.

In some such examples, the portion of the PCR mixture within the thermal cycling zone subject to the three different temperatures phases implemented via the first sheet comprises less than about 5 percent (e.g. 4.5, 4.6, 4.7, 4.8, 4.9, 5.0, 5.1, 5.2, 5.3, 5.4, 5.5) of the overall volume of the PCR mixture. As further described later, in some examples the thermal cycling zone may comprise even smaller portions of the overall volume of the PCR mixture to which the three different temperature phases (caused by the first sheet 122) is applied. In some examples, at any given time, the remaining volume (e.g. about 95% or more) of the PCR sample volume within the PCR well is not subject to thermal cycling, i.e. has a temperature below at least the second temperature of the second phase.

As further described later, the portion of the PCR mixture subject to the three temperature phases caused by local heating via the first sheet may comprise other percentages lower than 5 percent of the overall volume of the PCR mixture.

Via such arrangements, the application of local heating via such a thermal cycling zone may dramatically reduce the time to complete a thermal cycle, which may be on the order ones of seconds of time, such as less than about 2 seconds (e.g. 1.9, 1.95, 2, 2.05, 2.1) in some examples. Such relatively short thermal cycles may be repeated up to hundreds of times until a sufficient number of amplicons (e.g. target nucleic acid sequences) is produced to achieve a desired limit of detection for the analyte of interest.

The relatively short thermal cycling times, in turn, may dramatically reduce the time to complete the full number of thermal cycles to perform a PCR test, which may be on the order of tens of minutes such as, but not limited to, 15 or 20 minutes. This short overall completion time stands in sharp contrast to some other PCR tests, which take considerably longer to perform.

To accentuate the efficiency of such local heating, in some examples, magnetic attraction may be used to draw components within the PCR mixture in the PCR well into the thermal cycling zone. Accordingly, in some examples, the PCR well is to receive the PCR mixture, comprising beads, into the thermal cycling zone. In some such examples, via gravity and/or application of magnetic force, the beads travel into, and remain present within, thermal cycling zone.

In some such examples, the PCR mixture may comprise magnetic beads which are functionalized with the single-stranded nucleic acids (e.g. target nucleic acid sequence). In some examples, the functionalized beads may be paramagnetic or may be superparamagnetic, in some examples. In some examples, the thermal cycling zone Z also may comprise a location to which magnetic forces draw the functionalized beads so that the components of the PCR mixture to be subjected to the denaturation temperature within the thermal cycling zone may arrive more quickly, which in turn increases the rate of amplification of the target nucleic acid sequence. This arrangement, in turn, heightens the speed, accuracy, and reliability of the PCR test.

With these examples of pulse-controlled amplification in mind, it will be understood that the significantly increased thermal control by locally heating a PCR mixture via a wall of the PCR well via at least some examples of the present disclosure may significantly enhance the precision of the local heating and rapid cooling which occurs in relation to a thermal cycling zone when performing a PCR test. In particular, in some examples, such as a three-phase PCR test via pulse-controlled amplification, the significantly increased thermal differential between a heat sink and the PCR mixture within the PCR well helps to rapidly implement a second temperature phase after the initial pulse of first temperature and/or a third temperature after the second temperature phase. This significantly increased thermal differential also may help accurately and rapidly implement, and switch between, the different second and third temperature phases during the thermal cycle.

In one aspect, the significantly increased accuracy in temperature sensing, which supports significantly increased temperature control, etc. relating to a single PCR well also may contribute to consistency among multiple wells of a well plate or well chip, which may contribute to overall accuracy and reliability on an overall basis for the entire well plate.

With these various examples in mind, at least some further examples may comprise a device comprising a well and a control portion, wherein the well is to receive a polymerase chain reaction (PCR) mixture within an interior partially defined by a first wall, which comprises a heating first sheet. The control portion is to determine a first temperature of a first portion of the first sheet exposed to the interior, based on monitoring a resistance of the first sheet to determine an average temperature of the first sheet and based on a correction model, the correction model representing thermal behavior of at least one structure further defining the well and in thermal relation with at least a second portion of the first sheet, wherein the correction model is based on at least one model digital filter accounting for at least one of a geometry, a material, and a resistance sensitivity of at least the first sheet, and a geometry of the at least one structure further defining the well.

The control portion is to determine the first temperature of the first portion of the first sheet, by generating via the correction model, a temperature correction value to be applied to the average temperature of the first sheet, wherein the correction model at least partially represents the thermal behavior according to at least one of: a heating power of the first sheet during heating; and a cooling power associated with the at least one structure, which comprises a heat sink in indirect thermal contact with the first sheet.

In some examples, the correction model is to at least partially represent the thermal behavior by digitally filtering the heating power and the cooling power. Digitally filtering the heating power is to remove at least one of: a first temperature change occurring slower than an overall thermal response of the well; and a second temperature change faster than local time constants of the first sheet. Digitally filtering the cooling power is to remove at least one of: a third temperature change occurring slower than the overall thermal response of the well connected to a heat sink; and a fourth temperature change faster than local time constants of the well.

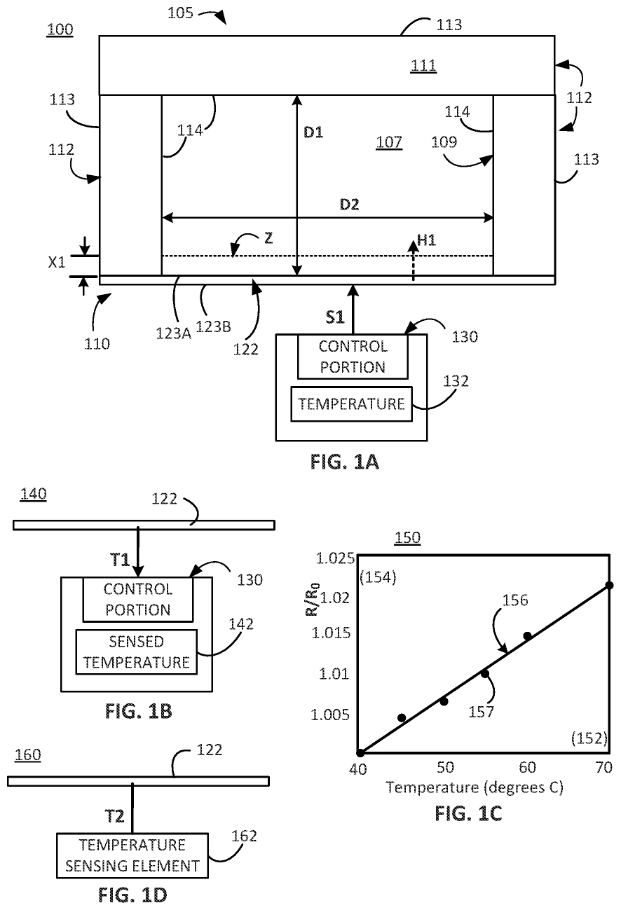

These examples, and additional examples, are described below in association with at least FIGS. 1-14.

FIG. 1A is a side sectional view of a testing device 100 comprising an example well 105 for performing a polymerase chain reaction (PCR) test. As shown in FIG. 1A, the PCR well 105 comprises first wall 110 and second walls 112, which together define an interior 109, which in turn comprises a closed container, e.g. sealed volume. In some examples, depending on the particular construction of the testing device 100, one of the second walls 112 may sometimes be referred to as a cover, as represented by indicator 111. Each second wall 112 comprises an external surface 113 and opposite internal surface 114. In some examples, the external surface 113 of each wall 112 may form part of, or be joined to, another part of a well chip structure such that the walls 112 sometimes may be referred to by other names reflective of this relationship. For instance, the walls 112 may sometimes be referred to as side portions.

In some examples, the second walls 112 comprise a dielectric material. In some such examples, the second walls 112 may comprise a polymer material, such as (but not limited to) a cyclic olefin copolymer (COC) material. In some examples, the polymer material may comprise polyethylene, polypropylene, polycarbonate, polymethylmethacrylate (PMMA), an SU-8 material, and the like. In one aspect, the SU-8 material may comprise an epoxy-based negative photoresist material. In some examples, the cover 111 (one example second wall 112) may comprise a transparent material.

In some examples, the first wall 110 comprises first sheet 122 (e.g. layer), which comprises a first surface 123A and an opposite second surface 123B. The first surface 123A is exposed to and partially defines an interior 109 of PCR well 105, such that the second walls 112 and the first sheet 122 of the first wall 110 define the interior 109 of the PCR well 105. The interior 109 defines a receptacle to receive and then sealingly contain a polymerase chain reaction (PCR) mixture 107. In some examples, the interior 109 of the PCR well 105 may comprise a volume of about 20 to about 30 microliters, and in some examples 25 microliters. In some such examples, a volume of the PCR mixture 107 comprises approximately the same volume as the interior 109 of PCR well 105 such that no air or minimal air is present with the PCR mixture 107 within the interior 109 of the PCR well 105.

In some examples, the PCR mixture 107 may comprise at least some of substantially the same features and attributes of the previously described PCR mixtures, which comprise at least a target genetic material (e.g. target nucleic acid sequence) and a PCR master mix, with the target genetic material corresponding to the analyte of interest. The target genetic material may also sometimes referred to as an amplicon in the context of the nucleic acid amplification testing.

The first sheet 122 (of the first wall 110) may comprise an electrically activatable heating element suitable to generate heat within the PCR well 105 for performing the PCR test. In some examples, the first sheet 122 may comprise an electrically conductive material, and in some instances may be referred to as a heating foil or a heating sheet. Upon application of a signal, this electrically conductive material will generate power (P) depending on its resistivity (R) and current (I) where P=I\hat{ }2×R. Accordingly, in some instances, the first sheet 122 also may sometimes be referred to as being an electrically resistive sheet.

In some examples, the first sheet 122 may comprise a thickness (T1) of about 10 to about 50 microns, a thickness of about 15 to about 40 microns, or a thickness of about 20 to about 30 microns, and in some examples, a thickness of about 25 microns. In some examples, the first sheet 122 may comprise a material selected from the group of stainless steel, brass, titanium, tantalum, tungsten, aluminum, copper, platinum, gold, silver, zinc, indium tine oxide (ITO), and combinations thereof. In some such examples, the first sheet material comprise a stainless steel 304 material and in some other examples, the first sheet material comprises a stainless steel 306 material.

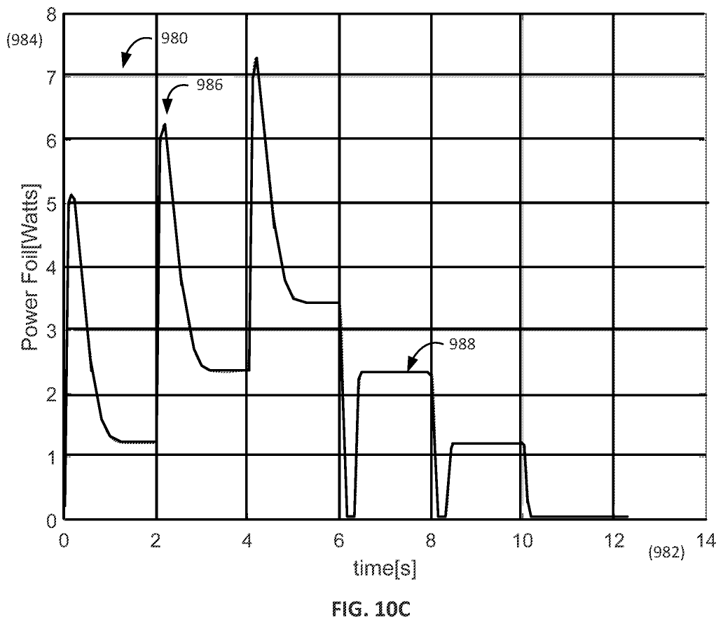

In some examples, at least some aspects of operation of, and/or monitoring of, the device 100 may be implemented via an example control portion 130 in FIG. 1A. For instance, control portion 130 may monitor and/or control the application of power from the signal source to the first sheet 122 (e.g. heating element) forming part of the first wall 110, which in turn may control at least some of the generation and application of heat within the PCR well 105. In some such examples, the control portion 130 may implement such monitoring and/or control via a temperature parameter 132. In some examples, the temperature parameter 132 may comprise a temperature phase parameter. However, in some examples, the temperature parameter 132 may comprise a temperature sensing parameter, which may sense a temperature according to at least some examples of the present disclosure in accordance with at least FIGS. 10A-11C, 12, and 14, in which a correction model may be applied to provide increased accuracy in temperature sensing of the PCR mixture 107 (at least a portion within a thermal cycling zone Z). Moreover, in some examples, the temperature parameter 132 may comprise a temperature sensing parameter, which may sense a temperature according to at least some examples of the present disclosure in accordance with FIGS. 1B-1C.

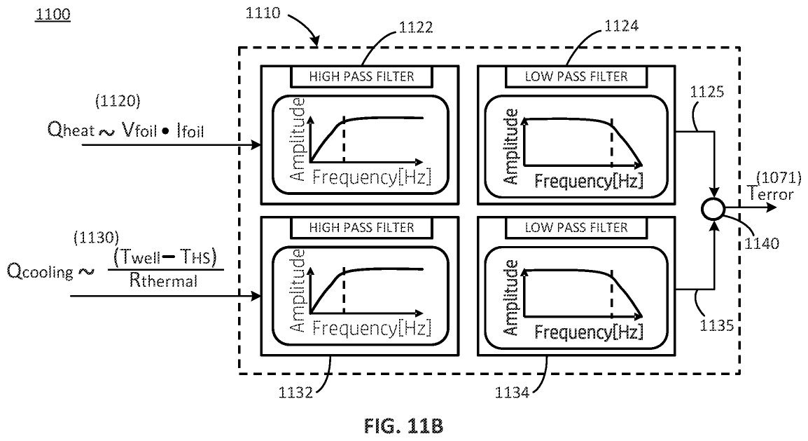

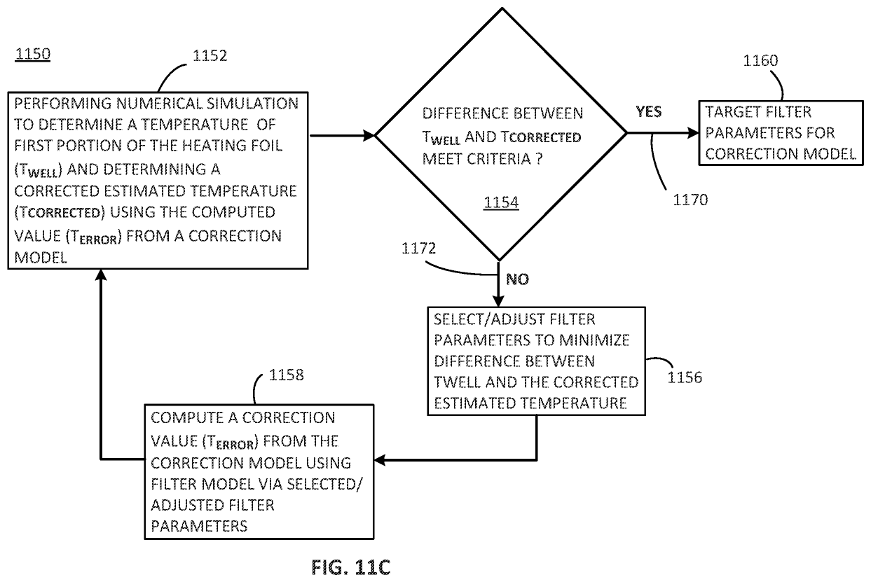

In some examples, the control portion 130 in FIG. 1A may comprise at least some of substantially the same features and attributes as, and/or comprise an example implementation of, the example control portion 1300 in FIG. 13A and/or operations engine 1200 in FIG. 12.

In some examples, the control portion 130 is to cause, via an electrical signal, the first sheet 122 to generate heat in a thermal cycling zone within the interior in close thermal proximity to the first sheet 122 in different phases operating in sequence at: a first temperature comprising at least 90° Celsius; a second temperature comprising at least about 25° C. less than the first temperature; and a third temperature comprising at least about 5° C. greater than the second temperature and at least about 15° C. less than the first temperature.

Further details regarding such heating and temperature control of a first sheet 122 and portion(s) of a PCR mixture 107 will be described below in the various examples of the present disclosure in association with at least FIGS. 1B-14.

FIG. 1B is a diagram including a side view schematically representing an example device 140 and/or example method of sensing a temperature of a first sheet 122. In some examples, the example arrangement in FIG. 1B may comprise at least some of substantially the same features and attributes as, and/or an example implementation of, the example of FIG. 1A. As shown in FIG. 1B, via at least the sensed temperature parameter 142, the control portion 130 is to sense a temperature of the first sheet 122 such as, but not limited to, by determining changes in a resistance of the first sheet 122, as represented by arrow T1. Because the first sheet 122 is directly exposed to, and in direct thermal relation with, the PCR mixture 107 in the PCR well 105, a temperature of the PCR mixture 107 will directly correspond to the temperature of the first sheet 122. Accordingly, a determination of changes in resistance of the first sheet 122 throughout the course of a PCR test, such as during the different temperature phases, provides a corresponding indication of the temperature of the first sheet 122, and therefore an indication of the temperature of a portion of PCR mixture 107 within the thermal cycling zone (Z in FIG. 1A) in close thermal proximity to the first sheet 122.

With this relationship in mind, FIG. 1C is a graph 150 schematically representing an experimentally determined relationship between a resistance changes of a heating foil, such as first sheet 122, relative to a temperature of the heating foil. As shown in FIG. 1C, in general terms a line 156 plotted relative to the measured data points 157 (e.g. black dots) reveals a generally linear relationship between the change in resistance (R/R₀) represented along Y-axis 154 and the temperature (in degrees Celsius) of the first sheet 122 represented along X-axis 152. In one aspect, in this experimental example the heating foil comprised a 304 stainless steel material having a thickness of about 25 microns.

Accordingly, before, during, or after the different temperature phases of a PCR test, the example device may continually (or periodically) track a temperature of the first sheet 122 (and therefore a temperature of the portion of the PCR mixture 107 within the thermal cycling zone) by tracking changes in resistance of the first sheet 122.

As previously noted, the sensed temperature of the first sheet 122 may be used by the control portion 130 to adjust parameters of a power signal applied to cause the first sheet 122 to generate heat for application to the portion of the PCR mixture within the thermal cycling zone (Z).

In some examples, the example PCR well 105 and/or the sensed temperature parameter 132 of control portion 130 may be implemented according to at least some examples of the present disclosure in accordance with at least FIGS. 10A-11C, 12, and 14, in which a correction model may be applied to provide increased accuracy in temperature sensing of the PCR mixture 107 (at least a portion within a thermal cycling zone Z).

FIG. 1D is a diagram including a side view schematically representing an example device 160 and/or example method of sensing a temperature of a first sheet 122 using an external sensor instead of or in addition to measuring a resistivity of first sheet 122. In some examples, the example arrangement in FIG. 1D may comprise at least some of substantially the same features and attributes as, and/or an example implementation of, the example of FIGS. 1A-C, except comprising a temperature sensing element 162 other than first sheet 122. As shown in FIG. 1D, the example device 160 comprises a temperature sensing element 162 coupled relative to the first sheet 122 in order to sense the temperature of the first sheet 122. In some examples, the temperature sensing element 162 may comprise an infrared (IR) sensor, a thermocouple, thermistor, and other temperature sensing modality. However, it will be understood that in some examples, that at least a portion the temperature sensing element 162 may become part of the thermal mass in which at least portions of the first sheet 122 is incorporated, such that a correction model may be used in some examples to account for those relationships in order to determine a temperature which accurately corresponds to the temperature of the PCR mixture 107 within the thermal cycling zone (Z).

With further reference to at least FIG. 1A, in some examples, the different temperature phases by which a PCT test is performed via the example device 100 may comprise a first phase operating at the first temperature for a first duration of no more than about 10 milliseconds (e.g. 9.5, 9.6, 9.7, 9.8, 9.9, 10, 10.1, 10.2, 10.3, 10.4, 10.5 milliseconds), a second phase operating at the second temperature for a second duration of at least about 500 milliseconds (e.g. 498, 499, 500, 501, 502 milliseconds), and a third phase operating at the third temperature for a third duration of at least about 500 milliseconds (e.g. 498, 499, 500, 501, 502 milliseconds). In some such examples, a second phase operating at the second temperature for a second duration comprise at least about 300 (e.g. 248, 249, 250, 251, 252 milliseconds) milliseconds, and a third phase operating at the third temperature for a third duration comprises at least about 300 milliseconds. Via such arrangements, a total duration of a thermal cycle may comprise less than about 1 to 1.5 seconds, although the total duration may be greater in some examples, as previously described and as further described below.

The first phase may sometimes be referred to as a first temperature phase, the second phase may sometimes be referred to as a second temperature phase, and/or the third phase may sometimes be referred to as a third temperature phase. In some other examples, the first phase may have a first duration of no more than about 15 (e.g. 14.15, 14.6, 14.7, 14.8, 14.9, 15, 15.1, 15.2, 15.3, 15.4, 15.5) milliseconds, no more than about 20 (e.g. 19.5, 19.6, 19.7, 19.8, 19.9, 20, 20.1, 20.2, 20.3, 20.4, 20.5) milliseconds, and so on. In some other examples, the second phase may comprise a second duration of at least about 550 milliseconds (e.g. 548, 549, 550, 551, 552 milliseconds), at least about 600 milliseconds (e.g. 598, 599, 600, 601, 602 milliseconds), and so on. In some other examples, the third phase may comprise a third duration of at least about 550 milliseconds (e.g. 548, 549, 550, 551, 552 milliseconds), at least about 600 milliseconds (e.g. 598, 599, 600, 601, 602 milliseconds), and so on.

Via such arrangements, a total duration of a thermal cycle may comprise less than about 1.75 seconds (e.g. 1.73, 1.74, 1.75, 1.76, 1.77), less than about 2 seconds (e.g. 1.98, 1.99, 2, 2.01, 2.02), less than about 2.25 seconds (e.g. 2.23, 2.24, 2.25, 2.26, 2.27), and so on.

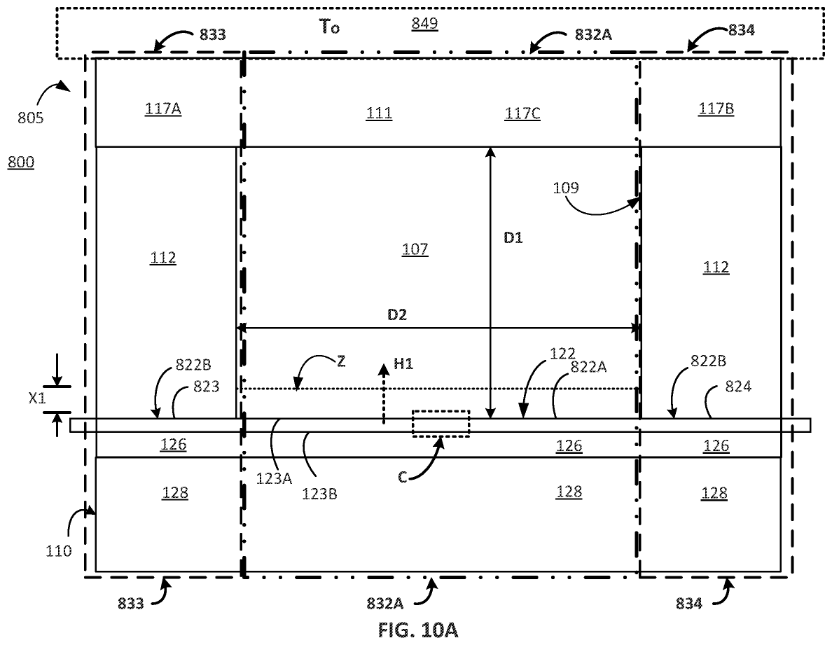

In some examples, the control portion 130 is to implement the heating via a pulse power mode to cause heating of the PCR mixture within a first temperature range including the first temperature; and a continuous power mode to cause heating of the PCR mixture within a second temperature range including the second temperature and within a third temperature range including the third temperature. In some such examples, the pulse power mode and/or the continuous power mode may be implemented via the pulse parameter 1212 and continuous parameter 1214, respectively, of the heating engine 1210 of the operations engine 1200 in FIG. 12. Moreover, in addition to or without the heating engine 1210, in some examples, the pulse power mode and/or continuous power mode may be implemented via a power modulator 562 of a signal source 560, as described later in association with at least FIG. 8.

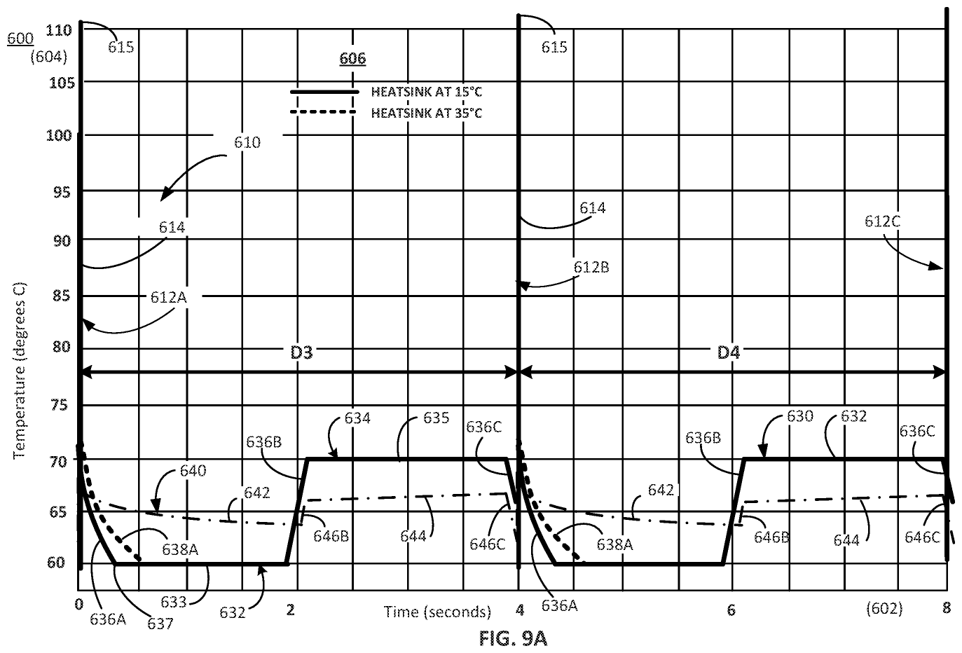

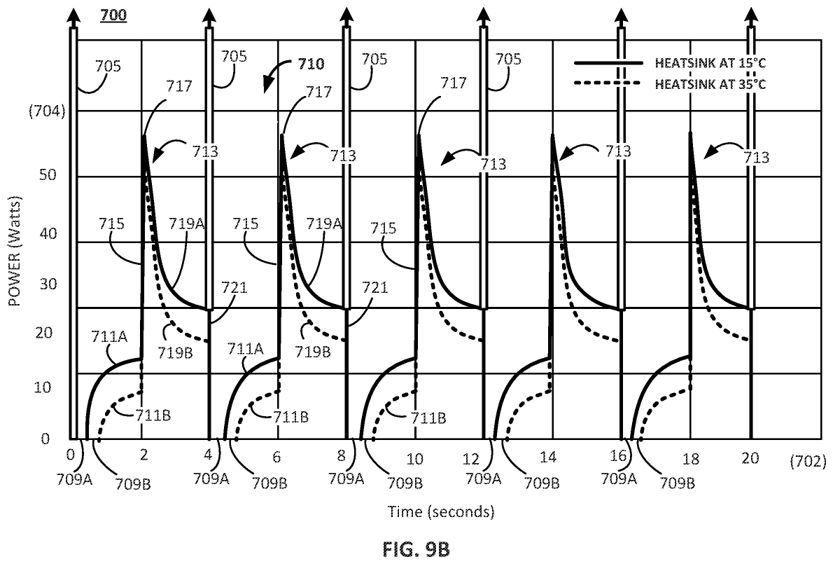

In some examples, the heating first sheet 122 comprises the sole heating element used to generate heat for application to the PCR mixture 107 during the thermal cycling. In some such examples, the modulation of the temperature of the first sheet 122 (and therefore a portion of the PCR mixture 107) may be enhanced via use of heat sink (e.g. 430 in FIGS. 5-6), which is operated at temperature substantially less than a temperature of the first sheet 122 such as, but not limited to, one of the three different temperatures of the respective first, second, and third phases. In some of these examples, the heat sink temperature is substantially less than a temperature in the second phase (e.g. annealing) of the thermal cycles.

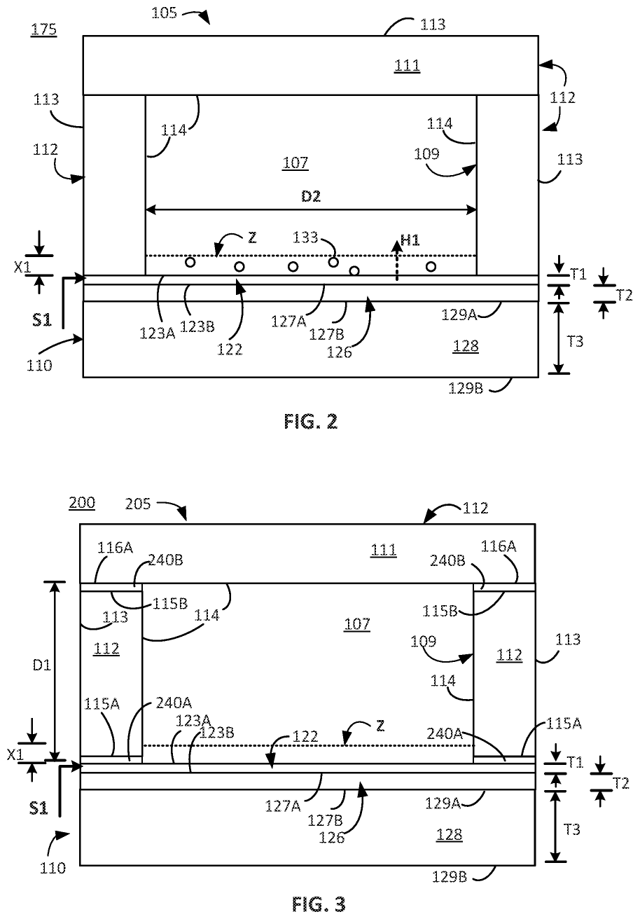

In some examples, and as later further described in association with at least FIGS. 2 and 5-6, the first wall comprises at least a second sheet connected relative to the heating first sheet and to receive removable engagement from a heat sink, and wherein the heat sink is a substantially constant fourth temperature, which is at least about 15° C. less than at least the second temperature (of the heating first sheet 122). In some examples, a temperature of the heat sink (e.g. fourth temperature) is substantially less than the temperature of the heating first sheet. Numerous further aspects regarding the heat sink and its relation to the heating by the first wall of the PCR well are further described in association with at least FIGS. 5-9C.

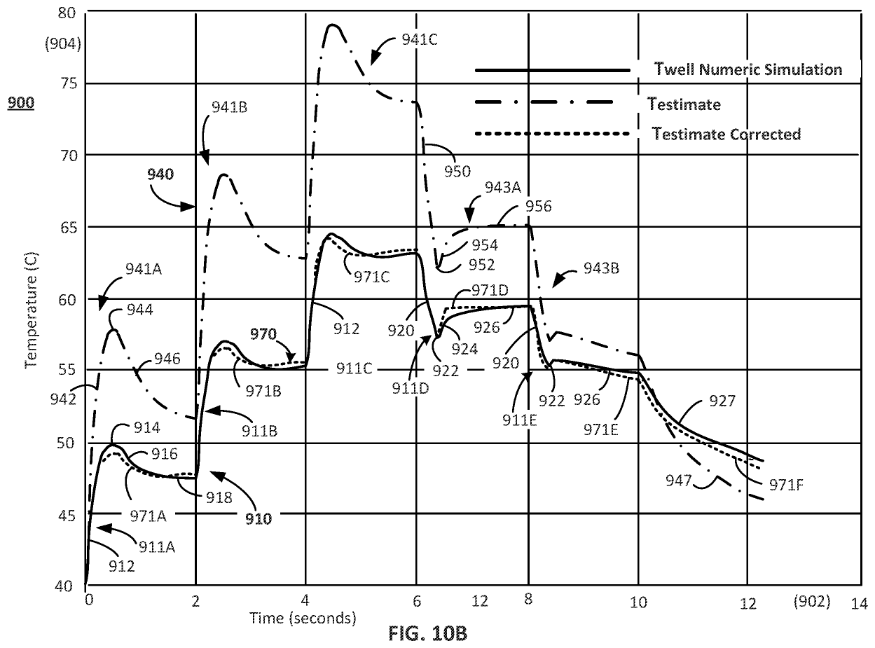

In some examples, the above-mentioned “substantially less” difference (between the heat sink temperature and the temperature of the first sheet 122) comprises a difference of at least about 25 (e.g. 24.5, 24.6, 24.7, 24.8, 24.9, 25, 25.1, 25.2, 25.3, 25.4, 25.5) percent less, at least about 30 (e.g. 29.5, 29.6, 29.7, 29.8, 29.9, 30, 30.1, 30.2, 30.3, 30.4, 30.5) percent less, at least about 35 (e.g. 34.5, 34.6, 34.7, 34.8, 34.9, 35, 35.1, 35.2, 35.3, 35.4, 35.5) percent less, at least about 40 (e.g. 39.5, 39.6, 39.7, 39.8, 39.9, 40, 40.1, 40.2, 40.3, 40.4, 40.5) percent less, at least about 45 (e.g. 44.5, 44.6, 44.7, 44.8, 44.9, 45, 45.1, 45.2, 45.3, 45.4, 45.5) percent less, at least about 50 (e.g. 49.5, 49.6, 49.7, 49.8, 49.9, 50, 50.1, 50.2, 50.3, 50.4, 50.5) percent less, at least about 55 (e.g. 54.5, 54.6, 54.7, 54.8, 54.9, 55, 55.1, 55.2, 55.3, 55.4, 55.5) percent less, at least about 60 (e.g. 59.5, 59.6, 59.7, 59.8, 59.9, 60, 60.1, 60.2, 60.3, 60.4, 60.5) percent less, at least about 65 (e.g. 64.5, 64.6, 64.7, 64.8, 64.9, 65, 65.1, 65.2, 65.3, 65.4, 65.5) percent less, at least about 70 (e.g. 69.5, 69.6, 69.7, 69.8, 69.9, 70, 70.1, 70.2, 70.3, 70.4, 70.5) percent less, at least about 75 (e.g. 74.5, 74.6, 74.7, 74.8, 74.9, 75, 75.1, 75.2, 75.3, 75.4, 75.5) percent less. For example, in an example of at least about 40 percent less, the heat sink temperature may comprise about 35 degrees Celsius. In an examples of about 75 percent less, temperature may comprise about 15 degrees Celsius. At least FIGS. 9A-9C provide further details regarding aspects of, and operation according to, such example heat sink temperatures (e.g. 35 degrees Celsius, 15 degrees Celsius).

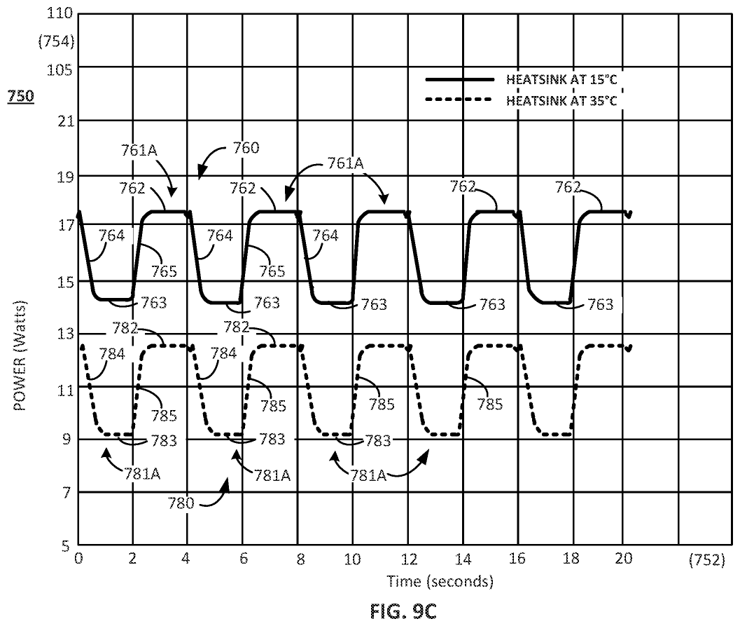

In some examples, the term substantially less (with regard to the difference between the temperature of the first sheet 122 and the temperature of the heat sink 430) may sometimes be referred to as the heat sink 430 being substantially cooler than the first sheet 122, such as in context of operation of the heat sink as described in association with at least FIGS. 6-7B.

In some examples, the PCR mixture 107 may comprise such PCR mixtures suitable for performing pulse-controlled amplification (PCA)-type polymerase chain reactions. Accordingly, the PCR mixture may sometimes be referred to as a PCA-PCR mixture.

In some examples, the thermal cycle for a polymerase chain reaction (PCR), according to a pulse-controlled amplification method, may be triggered by applying an initial current pulse of between about 20 Volts to about 60 Volts, and having a duration of about 0.3 to about 2 milliseconds. In some such examples, the initial current pulse may comprise about 40 Volts with a pulse duration of about 1 millisecond. In some such examples, the current pulse may comprise on the order of 100 amps, such as 105 amps. It will be understood that the various above-identified example values of initial current pulse parameters may be used to achieve a target temperature rise at the surface of the first sheet 122 of about 25 to about 35 degrees Celsius, which may generated by a net heat flux of about 1 to about 2.5 MWatts/m\hat{ }2 applied for about 1 milliseconds.

With this in mind, it will be understood that prior to application of the first heating pulse in PCA-PCR test, in some examples the first sheet 122 may apply heat to bring and maintain the PCR mixture 107 within the PRC well 105 to a target starting temperature. With this in mind, the above-described target temperature rise of about 25 to about 35 degrees Celsius would bring the temperature of the PCR mixture 107 from a starting temperature of about 65 to about 75 degrees Celsius (e.g. the elongation temperature, in some examples) to a temperature of at least about 90 (e.g. 89.5, 89.6, 89.7, 89.8, 89.9, 90, 90.1, 90.2, 90.3, 90.4, 90.5) degrees Celsius, which is sufficient to subject a portion of the PCR volume within the thermal cycling zone Z to the first target temperature for a first phase (e.g. denaturation) in a thermal cycle of the PCA-type PCR test. In some examples, the first temperature phase may comprise an upper limit such as, but not limited to, about 100 (e.g. 99.5, 99.6, 99.7, 99.8, 99.9, 100, 100.1, 100.2, 100.3, 100.4, 100.5) degrees Celsius.

It will be understood that the above-identified parameters may vary somewhat depending on a size of the PCR well 105, volume of the PCR mixture 107, as well as the size, materials, and/or shape of the first sheet 122 (e.g. heating element) of the first wall 110 by which the heat is generated, etc. It will be understood that in some examples, the heat generated, maintained, etc. within the interior 109 of PCR well 105 by first sheet 122 also may be affected, by a heat sink (e.g. 430), as previously described and as further described in association with at least FIGS. 5-8C.

In some examples, the interior 109 of the PCR well 105 may comprise a height D1 of about 300 (e.g. 298, 299, 300, 301, 302) microns to about 1500 (e.g. 1495, 1496, 1497, 1498, 1499, 1500, 1501, 1502, 1503, 1504, 1505) microns.

As represented via dashed line Z in FIG. 1A, in some examples a zone in which the thermal cycling occurs may sometimes be referred to as a general thermal cycling zone which is within a predetermined distance X1 of the first surface 123A (of the first sheet 122) of the first wall 110 of the PCR well 105 through which the heat is generated and applied. In some examples, this distance X1 may correspond to, and sometimes be referred to as, being within a close thermal proximity to the first wall 110.

In some examples, the predetermined distance X1 may be expressed as a percentage of the height D1 of the interior 109 of the PCR well 105. In some examples, the percentage may comprise at least about 5 percent of the height D1, while in some other examples, the percentage may comprise at least about 6 (e.g. 5.8, 5.9, 6.0, 6.1, 6.2) percent, at least about 7 (e.g. 6.8, 6.9, 7.0, 7.1, 7.2) percent, at least about 8 (e.g. 7.8, 7.9, 8.0, 8.1, 8.2) percent, at least about 9 (e.g. 8.8, 8.9, 9.0, 9.1, 9.2) percent, or at least about 10 (9.8, 9.9, 10, 10.1, 10.2) percent.

In some of these examples in which the height D1 may comprise about 1500 microns (i.e. 1.5 millimeters) and the predetermined distance comprises about 5 percent of the height D1, then X1 would comprise about 75 microns.

Conversely, in some of these examples in which the height D1 may comprise about 300 microns (i.e. 0.3 millimeters) and the predetermined distance comprises at least about 5 percent of the height D1, then X1 would comprise about 15 microns.

In some examples, the predetermined distance X1 may be less than 5% of the height D1 of the interior 109 of the PCR well 105.

In some such examples, a width D2 of the interior 109 of the PCR well 105 may comprise about 4 millimeters, while in some other examples the width D2 may comprise between about 1 millimeter and about 10 millimeters. In some examples, the width D2 may correspond to a diameter if the well 105 has a circular cross-sectional shape or correspond to a greatest cross-sectional dimension of the well 105 comprises a non-circular cross-sectional shape.

In some examples, the general thermal cycling zone Z also may comprise a location to which magnetic forces draw magnetic beads (e.g. functionalized with single-stranded nucleic acids as previously described) to heighten the effectiveness of the pulse-controlled amplification of the PCR process. One example implementation is later show in association with at least FIG. 2.

With further reference to FIG. 1A, upon receiving a signal (S1) from signal source (e.g. 560 in FIG. 8) via the control portion 130, the first sheet 122 generates heat (represented via directional arrow H1) for application to portion of the PCR mixture 107 within the thermal cycling zone (Z). While FIG. 1A depicts a single directional arrow H1, it will be understood that the heat H1 may be generated and applied across and along substantially the entire first surface 123A of first sheet 122.

As previously noted, in some such examples, the portion of the PCR mixture 107 in the thermal cycling zone Z which is subjected (via local heating by first sheet 122) to one of the first, second, or third temperatures of the three different phases of the thermal cycle may comprises less than about 5 percent (e.g. 4.8, 4.9, 5.0, 5.1, 5.2) of the overall volume of the PCR mixture 107. In some examples, the portion of the PCR mixture 107 within the thermal cycling zone Z subject to the first, second, or third temperature phases comprises less than about 4 percent (e.g. 3.8, 3.9, 4.0, 4.1, 4.2), less than about 3 percent (e.g. 2.8, 2.9, 3.0, 3.1, 3.2), less than about 2 percent (e.g. 1.8, 1.9, 2.0, 2.1, 2.2), or less than about 1 percent (e.g. 0.8, 0.9, 1.0, 1.1, 1.2) of the overall volume of the PCR mixture 107.

Accordingly, in some examples, a substantial volume of the PCR mixture 107 may be located vertically above the thermal cycling zone (Z). In some such examples, the substantial volume may comprise at least about 95 percent of the overall volume of the PCR mixture 107 such as when about 5 percent of the overall volume is within the thermal cycling zone Z.

Further details regarding such heating are described below in relation to at least the first sheet 122 of first wall 110 of the PCR well 105. Further details regarding the thermal cycling via application of heat are described below in association with at least FIGS. 9A-9C.

In some examples, one of the second walls 112 (e.g. 111) may comprise a material though which light may be transmitted to enable optical detection of output elements (e.g. fluorophores, etc.) resulting from the PCA-type, polymerase chain reaction. In general terms, a fluorophore may comprise a fluorescent chemical compound that can re-emit light upon light excitation. In some examples, such optical detection enables determining a presence, a quantity, or a concentration of a particular analyte (e.g. virus particle, other) to which the output element is attached (e.g. bonded). It will be understood that output elements (e.g. labels) other than fluorophores may be used in such optical detection examples.

With regard to these example dimensions, and other example dimensions throughout examples of the present disclosure, it will be understood that at least some components, spatial relationships, etc. in the Figures may be exaggerated (e.g. either made smaller or made larger) in scale for illustrative purposes, clarity, and/or simplicity.

While FIG. 1A depicts a single directional arrow H1, it will be understood that the heat H1 may be generated and applied relatively uniformly across (e.g. along) substantially the entire first surface 123A of first sheet 122. Similarly, upon instances in which a single arrow or just a couple of arrows (e.g. H1, H2, H3, etc.) are included in the FIGS to represent application of heat, thermal transfer, and the like, it will be understood that the heat (or general thermal transfer) may be applied relatively uniformly across (e.g. along) substantially the entire surface of the particular sheet, wall, etc. through which heat (or general thermal transfer) is occurring.

In some examples, the first sheet 122 may comprise a paramagnetic material or a ferromagnetic material. However, in some examples, the first sheet 122 may comprise a non-magnetic material at least to the extent that the material may be very weakly ferromagnetic or diamagnetic, and it is not intended to magnetically attract other objects such as beads to the first wall 110.

In some example, the PCR mixture 107 (and as part of the PCA-PCR process) may comprise beads which are functionalized with single-stranded nucleic acid. In some examples, the beads comprise a material and/or structure which is magnetic such that magnetic attraction of beads to the heating element 122 of the first wall 110 corresponds to attracting the nucleic acid strands (within the PCR mixture 107) into close thermal proximity to first wall 110. In some examples, the magnetic beads comprise paramagnetic beads and in some examples, the magnetic beads comprise superparamagnetic beads. In some such examples, the beads may be non-magnetic such that gravity alone may pull the functionalized beads toward and into the thermal cycling zone Z.

In one aspect relating to such examples, selective local heating of the PCR mixture 107 within the thermal cycling zone (Z) for all of the different temperature phases of the PCR test may enable more precise and accurate implementation of the respective first, second, and third temperature phases in PCR testing. This arrangement, in turn, contributes to testing which is more sensitive and able to detect lower quantities (or concentrations) of a particular analyte of interest (e.g. virus, other) and/or contributes to testing which can be performed more rapidly. In some examples, performing the second phase (e.g. annealing) and the third phase (e.g. elongation) of the thermal cycles at temperatures more directly within a target temperature range for the respective annealing and elongation phases may result in more efficient and/or effective annealing and elongation than when attempting to use a single temperature (in a two-phase PCR) to contemporaneously perform annealing and elongation.

FIG. 2 is a diagram including a sectional side view schematically representing an example testing device (and/or example testing method) including a well to perform polymerase chain reaction (PCR) testing. In some examples, the example device 175 in FIG. 2 may comprise at least some of substantially the same features and attributes as, and/or an example implementation of, the devices 100, 140, 160 as previously described in association with at least FIGS. 1A-1D. It will be understood that the device 175 may comprise the control portion 130, which is not shown in FIG. 2 (and/or other FIGS) for illustrative simplicity. In some examples, the first wall 110 may further comprise a second sheet 126 (e.g. layer), which comprises a first surface 127A and opposite second surface 127B. In some such examples the second sheet 126 may comprise a dielectric material and which acts to secure a third sheet 128 (of the first wall 110) relative to the first sheet 122.

In some examples, the second sheet 126 may comprise a thickness (T2) of about 30 microns to about 50 microns, while in some other examples the thickness (T2) may comprise about 50 microns to about 150 microns. The thickness may depend on the type of material forming the second sheet 126, as further described below.

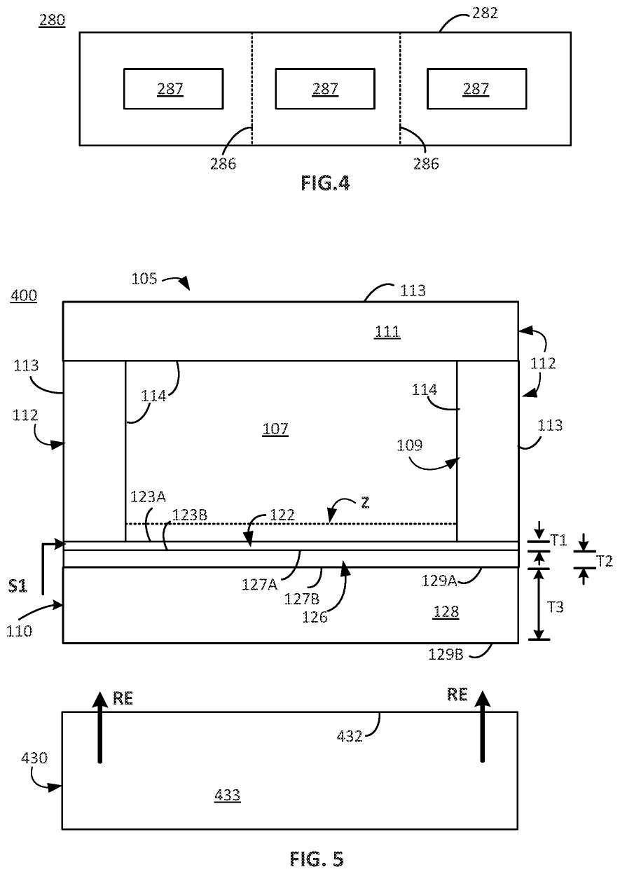

In some examples, the second sheet 126 comprises an adhesive layer, such as a pressure sensitive adhesive (PSA) layer, to secure the third sheet 128 relative to the first sheet 122. In some examples, the material of the second sheet 126 may comprise acrylic adhesive materials. In some of these examples, the second sheet 126 may comprise a thermal bonding adhesive, such as but not limited to: a Pyralux®-based material from DuPont de Nemours, Inc. of Wilmington, Delaware; and a FastelFilm material obtainable from Fastel Adhesives and Substrate Products via www.fasteladhesives.com; and the like. In some examples, the second sheet 126 may comprise a pressure sensitive adhesive comprising at least some of substantially the same features and attributes as one of the pressure sensitive adhesives for layers 240A, 240B, as later described in association with at least FIG. 3.

In some examples, the first wall 110 comprises the third sheet 128, which includes a first surface 129A facing and secured to the second surface 127B of the second sheet 126. The third sheet 128 also comprises an opposite second surface 129B to be releasably secured relative to a heat sink (e.g. 430), as further described later in association with at least FIGS. 5-6.

In some examples, the third sheet 128 may comprise a material which is made of the same material or similar material as the material from which second walls 112 are formed. Accordingly, in some examples, the third sheet 128 may comprise a polymer material, such as (but not limited to) a cyclic olefin copolymer (COC) material. In some examples, the polymer material may comprise polyethylene, polypropylene, polycarbonate, polymethylmethacrylate (PMMA), and the like.

Accordingly, in at least some examples, the third sheet 128 comprises a dielectric material, which acts an electric barrier to prevent electrical communication or contact between the electrically conductive first sheet 122 and other non-dielectric materials such as, but not limited to, materials from which the heat sink 430 may be formed. Moreover, as further described below, the third sheet 128 also may act as a thermal barrier.

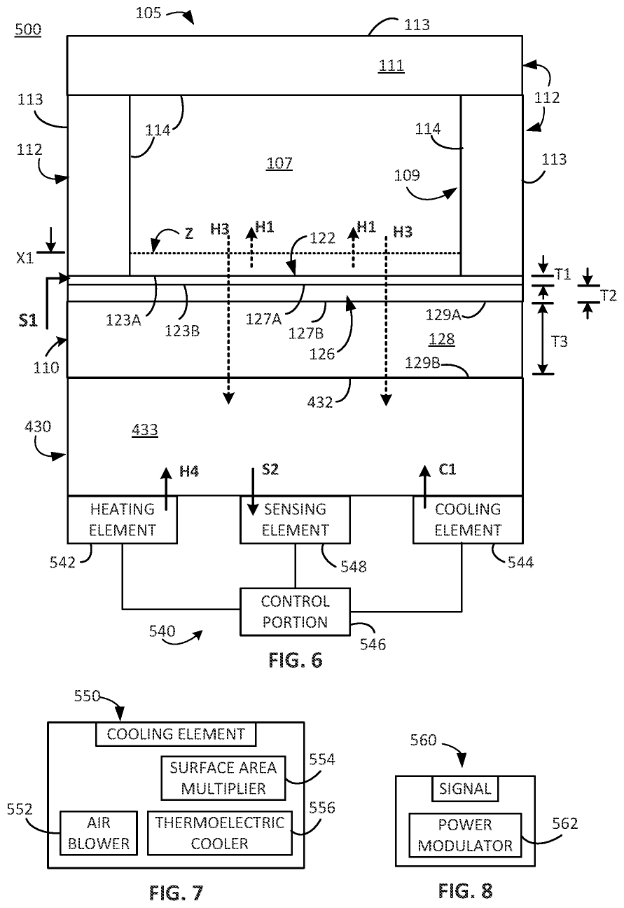

In some examples, the third sheet 128 may comprise a thickness (T3 in FIG. 2) of about 150 microns to about 500 microns, and in some examples a thickness of about 250 to about 400 microns. In some examples, the thickness (T3 in FIG. 2) may comprise about 300 microns. In one aspect, the thickness provides a mechanical stiffness sufficient to resist or prevent deformation of the first sheet 122 (e.g. heating element) of the first wall 110, which may comprise a relatively thin metal foil (e.g. thickness T1 of 10 to 50 microns) in some examples. Accordingly, this arrangement may make the PCR well 107 more robust during physical handling, thereby helping to ensure the accuracy and reliability of PCR testing due to the enhanced physical integrity of the structure defining the PCR well, including its heating components, thermal transfer components, and/or other components. Among other forms of physical handling, upon the PCR well 105 (e.g. a consumable cartridge) becoming releasably secured relative to a heat sink (e.g. 430 in FIGS. 3A-4), the significant thickness and relative rigidity of the third sheet 128 (of the PCR well 105) may help to maintain the ability of relatively high thermal transfer by helping to prevent bending, buckling, etc., which might otherwise contribute to undesired thermal gaps between the PCR well 105 and the heat sink.

In some examples, the third sheet 128 also acts as a thermal barrier to reduce immediate thermal influences on the first sheet 122 and the PCR mixture 107 from external sources such as, but not limited to, the heat sink 430. In particular, as previously noted, in view of the thermal mass of the heat sink (e.g. 430 in FIGS. 5-6) being substantially greater than a thermal mass of the heating first sheet 122, and the use of the heating first sheet 122 as the sole source to generate and apply heat within the thermal cycling zone (Z) in the interior of the PCR well 105, the third sheet 128 helps to thermally inhibit the thermal behavior of the heat sink from immediately, directly dominating the thermal behavior of the first sheet 122 and the PCR mixture 107. However, due to its relatively large thermal mass and general proximity to the first sheet 122 and PCR mixture 107, the heat sink (e.g. 430 in FIGS. 5-6) may indirectly thermally influence the temperature of the PCR mixture 107 and the first sheet 122 by acting as a ballast/counterweight to help modulate the temperature of the PCR mixture 107 at least during transitions between three different temperature phases of a PCR test controlled by the selective application of heat via the first sheet 122. Various aspects of the thermal mass and other properties of the heat sink are further described later in association with at least FIGS. 5-8C.

For instance, as further described later in association with at least FIGS. 9A-9C, by maintaining a heat sink at a constant temperature substantially less than the respective target temperatures of the different phases of the PCR process for PCR mixture (as controlled by local heating via the first sheet 122), the heat sink 430 may rapidly neutralize the thermal effects of the first sheet 122 rapidly upon cessation of initial heating pulse portion for first temperature phase or cessation of heating for third temperature phase. in acting as a ballast, the temperature of the first sheet 122 may be rapidly increased with the heat sink acting as a brake to help slow temperature change when rapid increase transitions to decreasing power (FIG. 8B) such as power curve segment for third temperature, and helps rapid transition from third temperature phase to a starting point prior to initial heating pulse portion of subsequent thermal cycle.

With this in mind, it is further noted that the later-described heat sink 430 (e.g. FIGS. 5-6) may comprise a material having a relatively high thermal conductivity, such as Aluminum or other materials with appreciable rigidity and suitable for rapid thermal transfer such as, but not limited to, for ease of cooling. In some examples, the heat sink may comprise a heat sink configuration including a cooling element (e.g. 544, 550 in FIGS. 6-7) to facilitate cooling from ambient air surrounding the heat sink (e.g. 552, 554 in FIG. 7) or via direct cooling (e.g. 556 in FIG. 7), such as further described in association with at least FIGS. 6-7.

In some examples, the second sheet 126 of the first wall 110 is directly connected to the first sheet 122 and the third sheet 128 of the first wall 110 is directly connected to the second sheet 126. In some such examples, this arrangement also may sometimes be described as the second wall omitting intervening structures between the second sheet 126 and the third sheet 128 and/or omitting intervening structures between the first sheet 122 and the second sheet 126. Moreover, as previously noted the first surface 123A of the first sheet 122 (e.g. heating element) is directly exposed to and partially defines the interior 109 of the PCR well 105, such that no intervening structure/layer is present between the heating element (e.g. first sheet 122) and the PCR mixture 107 within the interior 109 of the PCR well 105.

Further details regarding heating, thermal transfer, etc. involving the first wall 110 of the PCR well 105 are further described later in association with at least FIGS. 5-9C.

As shown in FIG. 2, in some example the PCR mixture 107 (and as part of the PCA-PCR process) may comprise beads 133 which are functionalized with a single-stranded nucleic acid, as previously noted in association with FIG. 1A. The beads 133 may comprise a material and/or structure which is magnetic such that magnetic attraction of beads to the heating element 122 of the first wall 110 corresponds to attracting the nucleic acid strands (within the PCR mixture 107) into close thermal proximity to first sheet 122 within the thermal cycling zone Z. However, in some examples, the beads may be non-magnetic such that gravity alone may pull the functionalized beads toward and into close proximity to first sheet 122 within the thermal cycling zone Z.

FIG. 3 is side sectional view schematically representing a testing device 200 including example PCR well 205. In some examples, the device 200 may comprise at least some of substantially the same features and attributes as the device 100 of FIG. 2. As shown in FIG. 3, device 200 comprises a reaction well 205 comprising at least some of substantially the same features and attributes as reaction well 105 (FIG. 2), except while further comprising adhesive layers 240A, 240B. The adhesive layer 240A is interposed between a first surface 115A of dielectric second walls 112 and a first surface 123A of first sheet 122 of first wall 110. Meanwhile, the adhesive layer 240B is interposed between a second surface 115B of dielectric second walls 112 and an inner surface 116A of the dielectric first wall 111.

In general terms, both of the adhesive layers 240A, 240B may comprise a pressure sensitive adhesive, which tends not to swell, which may have a low fluorescent level, which tends not to inhibit the polymerase chain reaction process, which seals well so as to resist the PCR mixture 107, and/or which otherwise helps to maintain desired operating conditions within the interior 109 of the PCR well 105.

In some examples, the adhesive layer 240A may comprise a pressure sensitive adhesive such as, but not limited to, an acrylic-based double-sided tape, which may comprise a clear thin plastic film coated on both sides with medical grade pressure-sensitive adhesive. In some such examples, the acrylic-based double-sided tape may comprise such tapes available from Adhesives Research, Inc. of Glen Rock, Pennsylvania.

In some examples, the adhesive layer 240B may comprise a pressure sensitive adhesive such as, but not limited to, an encapsulated silicone adhesive. In some examples, the encapsulated silicone adhesive may comprise adhesive sealing film polyolefin such as, but not limited to, those available from Innovative Laboratory Products of Phoenix, Arizona. In some other examples, the adhesive layer 240B may comprise a silicon-based, adhesive-coated polyolefin film such as those available from 3M of Saint Paul, Minnesota.

FIG. 4 is a side sectional view schematically representing an example testing device 280 (e.g. molecular testing device) comprising a plurality of reaction wells 287 arranged together within or on a common support structure 282. The dashed lines 286 represent a dividing line between adjacent wells 287, but such lines 286 do not necessarily correspond to a physical barrier, structure, etc. between adjacent wells 287. In some instances, the entire device may sometimes be referred to as a well plate or multi-well chip. In some examples, at least some of the wells 287 comprise at least some of substantially the same features and attributes including (or related to) the PCR well 105 in FIG. 4 and/or PCR well 205 in FIG. 2. With further reference to FIG. 4, it will be understood that testing device 280 is not limited to the number (e.g. 3) of wells 287 shown in FIG. 4, such that device 280 may comprise a greater number or lesser number of wells 287. Moreover, in some examples, testing device 280 may comprise wells 287 arranged in a two-dimensional array (e.g. 2×2, 3×2, 4×2, etc.). In some examples, the support 282 and/or individual wells 287 may comprise a portion of, and/or be in communication with, control portion (e.g. 1300 in FIG. 13A).

In some examples, the testing device 280 may comprise additional fluidic pathways, active and/or passive fluidic control components, etc., which in turn may comprise at least a portion of (or incorporate) the control portion (e.g. 1300 in FIG. 13A). Furthermore, in some examples the testing device 280 also may be removably connectable to a console, station, or the like to support performing, monitoring, evaluating, etc. tests in the wells 287, with the respective console (or station, other) comprising at least a portion of (or incorporate) the control portion (e.g. 1300 in FIG. 13A).

As shown in the diagram of FIG. 5, an example testing device 400 may comprise a PCR well 105 used with a heat sink 430 including a first surface 432, which is brought into releasable engagement (as represented by directional arrows RE) relative to the third sheet 128 of first wall 110 of the PCR well 105, with the resulting configuration shown in FIG. 6. In some examples, such releasable engagement may be maintained via a clamp or other mechanical fastening arrangements.

In some examples, the heat sink 430 may comprise a heat block 433, such as a cuboid shaped element made of a highly thermally conductive material, such as aluminum. In some examples, the aluminum material may comprise a thermal conductivity of between about 235 W/m·K and about 245 W/m·K, such as about 240 W/m·K.

In some examples, the heat block 433 may comprise a thermal mass on the order of about 50 to about 500 grams.

As further shown in the diagram of a testing device 500 in FIG. 6, with the heat sink 430 releasably engaged relative to the third sheet 128 of first wall 110 of PCR well 105, a pulse-controlled amplification, PCR reaction process may be performed. Via this arrangement, the heat sink is in thermal relation with (e.g. thermally coupled relative to) the first wall 120 of the PCR well 105, such as via the third sheet 128 (and second sheet 126) of the first wall 120. Via this arrangement, the heat sink 420 can thermally influence the electrically activatable first sheet 122 and at least a portion of the PCR mixture 107 within the thermal cycling zone in close proximity to the first sheet 122.

Further details regarding this general heating process is further described later in association with at least FIGS. 9A-9C.

FIG. 6 is a diagram of a testing device 500 including PCR well 105 releasably engaged relative to a heating block 430, with a heat sink control system 550 associated with the heating block 430. In some examples, the testing device 500 may comprise one example implementation of, and/or at least some of substantially the same features and attributes of, the example testing devices described in association with at least FIGS. 1-5, while further comprising heat sink control system 550.

As previously noted, via the signal S1 applied to the first sheet 122 of the first wall 110, heat H1 is applied in three different temperature phases within the thermal cycling zone (Z) in order to amplify reaction processes involving the polymerase chain reaction (PCR) mixture within the thermal cycling zone (Z) of the PCR well 105. An initial pulse portion of heating is used to cause a first phase (e.g. denaturation of nucleic acid strands) of a thermal cycle at a first temperature, after which rapid thermal transfer occurs from the first sheet 122 (and therefore the portion of the PCR mixture 107 within the thermal cycling zone) through the first wall 110 (including third sheet 128) into heat sink 430, as represented by dashed directional arrows H2. This rapid thermal transfer significantly contributes to lowering the temperature (e.g. cooling) of the PCR mixture 107 in PCR well 105 toward a target second temperature at which a second heating phase (e.g. annealing) is to occur. Thereafter, a third heating phase is caused by increased heating by the first sheet 122 to complete one of many thermal cycles within the thermal cycling zone in close thermal proximity to the first sheet 122. As previously described in introducing the examples of the present disclosure, at least a temperature of the heat sink 430 being substantially less (i.e. substantially cooler) than a temperature of the first sheet 122 enables the above-described rapid thermal transfer from the PCR mixture 107 to the heat sink 430 and/or influencing a temperature of the first sheet 122 during transitions between the second temperature phase and the third temperature phase, and transitions between the third temperature phase and the first temperature phase of a subsequent thermal cycle within the thermal cycling zone (Z).

With this in mind, further details regarding the heat sink (FIGS. 6-7) and/or further details regarding a temperature profile (FIG. 9A), a heating power signal profile (FIG. 9B), and/or a cooling power signal profile (FIG. 9C) are described below.

As further shown in FIG. 6, in some examples, the device 500 may comprise a heat sink control system 540, which may comprise a heating element 542, a cooling element 544, a control portion 546, and a temperature sensing element 548. In some examples, the temperature sensing element 548 may comprise a thermocouple, a thermistor, or an infrared sensor. In some examples, each of the heating element 542 and the cooling element 544 are connected to and/or otherwise incorporated into the heat sink 430 while in some examples, the temperature sensing element 548 is connected to and/or otherwise incorporated into the heat sink 430. Via operation of control portion 546, the temperature sensing element 558 may sense a temperature of the heat sink 430 (as represented by arrow S2), and using the sensed temperature, the control portion 546 may direct operation of the heating element 542 to apply heat (as represented via arrow H4) and/or of the cooling element 544 to apply cooling (as represented by arrow Cl) to maintain the heat sink 430 at a selected target temperature. Stated differently, in some examples, the control portion 546 may cause the heat sink 430 to exhibit a target temperature of the heat sink 430 via controlled operation of the heating element 542 and/or cooling element 544 based on the sensing of, via the temperature sensing element 558, a temperature of the heat sink.

However, it will be understood that in some examples, a presence of the ambient air (e.g. such as at 20 degrees Celsius) may be used to move the heat sink 430 to, or maintain the heat sink 430 at, a selected target temperature. Accordingly, the ambient temperature may be used in addition to, or instead of, the heating element 542 and/or the cooling element 544 to achieve, in cooperation with the temperature sensing element 558, the selected target temperature of the heat sink 430. For instance, in some of these examples such as when a selected target temperature of the heat sink 430 is about 35 degrees Celsius, cooling the heat sink 430 via the ambient air may be used in some instances (at least part of the time) without the use of cooling element 544 to achieve the selected target temperature.

In some such examples, via the heat sink control system 550, a temperature of the heat sink 430 may be maintained at or near a target temperature range, such as a target temperature which is substantially less than target temperatures of first sheet 122 at which a first phase, second phase and/or third phase of PCR process take place within the thermal cycling zone. As previously noted in association with at least FIG. 6, in one aspect by maintaining the heat sink 430 at a selectable target temperature (within a selectable target temperature range), the arrangement significantly contributes to a rapid thermal transfer of heat out of the PCR mixture 107 (as represented via directional arrow H3 in FIG. 6) after an initial pulse portion for a first phase, and then acts as a thermal counterbalance to enhance the precision and agility of changing the temperature of the PCR mixture 107 (in PCR well 105) between two different target temperatures for the second and third phases of thermal cycling, as represented by directional arrows H3 in FIG. 6.

In some examples, the heat sink control system 540 operates in order to maintain the heat sink 430 at a selectable constant target temperature (substantially less than at least the selectable target second temperature of the first sheet 122) despite intended changes between the second target temperature and the third target temperature of the first sheet 122. At least one example implementation of selectively varying a magnitude of power applied to the thermal control system, such as applied to at least the cooling element, is further described and illustrated in association primarily with FIG. 9C and also FIGS. 9A-9B.

Accordingly, in some examples, via the control portion 546 the heat sink control system 540 may be operated to maintain, as close as possible, the heat sink 430 at a selectable target temperature such as, but not limited to, 15 degrees Celsius, 35 degrees Celsius, or other selectable temperatures. Among other aspects, by providing the heat sink 430 at a temperature which is substantially less (i.e. substantially cooler) than a lowest temperature of the first sheet 122 (e.g. second target temperature in the second phase), a substantial temperature differential is maintained between the first sheet 122 and the heat sink 430, such that the temperature of the first sheet 122, after an initial pulse portion at a first target temperature will rapidly be cooled and after a short time period approach the second target temperature of the first sheet 122. At that time or after a short delay, the first sheet 122 may again begin heating to establish and maintain the first sheet 122 at the second target temperature 122, which in turn causes the PCR mixture 107 (within the thermal cycling zone) to exhibit the second target temperature within the thermal cycling zone (Z). Near or at the end of the second temperature phase, more power is applied to the first sheet 122 to increase heating of the first sheet 122 to raise the temperature of the first sheet 122 to a third target temperature to cause the third temperature phase within the thermally cycling zone (Z).

As previously noted, the substantially cooler heat sink 430 (e.g. having a temperature substantially less than a temperature of the first sheet 122) acts as a thermal counterbalance (as represented by directional arrows H3 in FIG. 6) on an on-goring basis to enhance the precision and agility of changing the temperature of the PCR mixture 107 (in PCR well 105) between two different target temperatures for the respective second and third temperature phases. Further details regarding this relationship will be described in association with at least FIGS. 9A-9C.

In some examples, the heat sink control portion 546 in the device 500 of FIG. 6 may comprise one example implementation of, and/or comprise at least some of substantially the same features and attributes as, the control portion 1300 in FIG. 13A.

With this arrangement in mind, at least some further examples of the cooling element 544 of FIG. 6 will be described.

FIG. 7A is a block diagram schematically representing an example cooling element 580, which may comprise at least some of substantially the same features and attributes as, and/or an example implementation of, the cooling element 554 in FIG. 6.

As shown in FIG. 7A, the cooling element 580 may comprise an air blower 582, a surface area multiplier 584, and/or a thermoelectric cooling element 586. In some examples, the air blower 582 may be positioned on, within, or near the heat sink 430 (block) to blow air over, around, and/or through the heat sink 430 (e.g. block) to cool the heat sink 430.

The surface area multiplier 584 may comprise a structure which is in thermal contact with the heat sink 430 (e.g. block 433) and which significantly increases a surface area which is exposed to the ambient air. In some examples, the surface area multiplier 584 may comprise a material which has a relatively high thermal conductivity such as, but not limited to, aluminum, with the surface area multiplier 554 comprising a set of fins, in some examples. In some such examples, the air blower 582 may be used in conjunction with the surface area multiplier 584 by blowing air over, around, and/or through the high surface area elements to enhance their cooling effect.

In some examples, the thermoelectric cooling element 586 (of the cooling element 580) may comprise a solid state thermoelectric cooling element such as, but not limited to, a Peltier-style cooling element. The thermoelectric cooling element 586 may be in thermal relation to the heat sink 430 (e.g. block 433), such as via direct contact when secured against the heat sink 430 alone and/or in a complementary manner with other example cooling elements.

In some examples, just one of, or a combination of, the air blower 582, surface area multiplier 584, and the thermoelectric cooling element 586 may be used to evacuate on the order of 10 Watts (e.g. 11 Watts) to maintain the heat sink 430 (including heat block 433) at 35 degrees Celsius.

In some examples, the heat sink 430 may comprise a thermal resistance of about 0.3 degrees Celsius/Watt. In this example, if the heatsink is kept at 35 C and needs to evacuate 11 W, the difference (e.g. delta T) between the temperature of the heatsink 430 and the cooling air (or active cooler) may comprise about 3.3 degrees Celsius.

With these example arrangements in mind, FIGS. 9A-9C illustrate several aspects of temperature control for an example testing device (and/or example method) for a PCR well, utilizing at least some of the examples of the present disclosure described in association with at least FIGS. 1-7.

FIG. 8 is a block diagram schematically representing an example signal source 560, which may comprise an example implementation of, and/or at least some of substantially the same features and attributes of, a signal source, as previously described in association with at least FIGS. 1A-7 for applying an electrical signal to first sheet 122 to generate heat. In some examples, the signal source 560 may comprise a power modulator 562. Among other aspects, the power modulator 562 may be used to selectively apply different amounts of power to the first sheet 122 to enhance implementing the three different temperature phases of thermal cycling. In some examples, a similar power modulator 560 also may be used to selectively apply different amounts of power to a cooling element 544 and/or a heating element 542 of the heat sink control system 540 to enhance the actions of the first sheet 122 in implementing the selected temperature profile.

In some of these examples, the power modulator 562 may comprise a pulse width modulator, a current intensity modulator, and/or other elements to control a voltage and current of a power signal.

At least some further aspects or effects of the power modulator 562 in producing a power signal are described in association with at least FIGS. 9A-9C.

FIG. 9A is a graph 600 which schematically represents a temperature profile 610 (y-axis 604) of a first sheet 122 (of a first wall 110 of a PCR well 105) plotted over time (x-axis 602) for a series of example thermal cycles 612A. 612B, and so on.

In one aspect, the temperature profile 610 plotted on graph 600 corresponds to thermal cycles 612A, 612B which were numerically simulated according an example device comprising at least some of substantially the same features as the examples of the present disclosure described in association with at least FIGS. 1A-7. In some such examples, a first sheet 122 (FIGS. 1A-7) of a first wall 110 of a PCR well 105 selectively, locally applies heat in all three phases of PCR process to modulate a temperature of a portion of the PCR mixture within the thermal cycling zone in the interior of the PCR well in close thermal proximity to the first wall 120. In at least some of these examples, this temperature modulation via local heating by the first sheet 122 (of first wall 110) may be enhanced via a heat sink in thermal relation to, and having a temperature substantially less (i.e. substantially cooler) than, the first sheet 122 of first wall 110. With this in mind, graph 600 provides further details regarding the heating phases applied via the first sheet 122 of the first wall 120 of the PCR well 105 and/or regarding thermal cycling in the examples of the present disclosure.

It will be further noted that the temperature profile 610 in graph 600 of FIG. 9A depicts the temperature profile 610 in a general manner for two different operating examples. In particular, as shown in the legend 606 in FIG. 9A, solid black lines of temperature profile 610 correspond to examples in which a heat sink 430 (releasably, thermally coupled relative to the PCR well 105) is continuously maintained at 15 degrees Celsius before and during the thermal cycles applied to the portion of the PCR mixture 107 within a thermal cycling zone (Z) (FIGS. 1A-6). Meanwhile, the dashed lines 638A in the graph 610 correspond to examples in which a heat sink 430 (releasably, thermally coupled relative to the PCR well 105) is continuously maintained at 35 degrees Celsius before and during the thermal cycles applied to the PCR mixture 107 within the thermal cycling zone (Z). It will be understood that the appearance of the dashed lines (e.g. 638A) in just a few locations in FIG. 9A reflects that, for the most part, the temperature profile of the first sheet 122 (and therefore the portion of the PCR mixture 10) is generally similar for either heat sink temperature (e.g. 15 degrees Celsius or 35 degrees Celsius) because the dominant factor determining the temperature of the PCR mixture 107 (e.g. portion within the thermal cycling zone Z) is the temperature of the first sheet 122 to which power is applied to locally heat just a portion of the PCR mixture within the thermal cycling zone (Z) in all three phases of each thermal cycle 612A, 612B, etc. To the extent that the different heat sink temperatures may produce some differences in the temperature profile 610, those differences are further described below in the context of describing the details of FIGS. 9A, 9B, and 9C.

The temperature profile 210 of the first sheet 122 (and therefore the portion of the PCR mixture 107 within the thermal cycling zone Z) during different phases of thermal cycling, as shown in FIG. 9A, will be considered in tandem with FIG. 9B, which schematically represents a power signal profile 710 applied for heating the PCR mixture via the first sheet 122 exposed to the PCR mixture 107 within the interior of the well. An initial description of the temperature profile 710 of the PCR mixture 107 in FIG. 9A will be directed to the black lines corresponding to the heat sink temperature of 15 degrees Celsius. Later, further comments will be provided regarding the dashed lines (e.g. 638A) corresponding to the heat sink temperature of 35 degrees Celsius.

With this in mind, as shown in FIG. 9A, in some examples each thermal cycle (e.g. 612A, 612B, etc.) has a duration of about 4 seconds (e.g. 3.8, 3.9, 4, 4.1, 4.2), and may generally comprise an initial rapid pulse of a first phase 614, a second phase (e.g. annealing) 632, and a third phase (e.g. elongation) 640. The first phase 614 includes an initial, pulse, which raises the temperature of the first sheet 122 from a starting temperature of about 65 (e.g. 64.8, 64.9, 65, 65.1, 65.2) degrees to about 75 (e.g. 74.8, 74.9, 75, 75.1, 75.2) degrees Celsius (e.g. the elongation temperature, in some examples) to a peak 615 above 90 degrees Celsius such as, but not limited to, 100 degrees Celsius. This initial pulse in the first phase 614 generally corresponds to an initial power signal portion 705 (as represented by hollow elongate rectangle) of one of the cycles 713 in the power signal curve 710 in FIG. 9B. In some examples, the initial signal portion 705 may comprise a magnitude of about 4000 Watts, which is beyond the scale of the Y-axis 704 in graph 700. However, it will be understood that the initial power signal portion 705 is included here to be juxtaposed relative to the power signal portions 711A, 715, 717, 719A which provide for a second phase (e.g. annealing) and a third phase (e.g. elongation). In this way, one can appreciate all the aspects (e.g. initial pulse and continuous power) of the power signal profile 710 for each complete thermal cycle (612A, 612B, and so on in FIGS. 9A and 713 in FIG. 9B).

With further reference to FIG. 9A, almost instantaneously after reaching peak 615 (via initial pulse portion 614), the temperature of first sheet 122 nearly instantaneously falls back to a temperature (e.g. near 68 to 70 degrees Celsius), which is near or within the range of the starting temperature (e.g. about 65 to about 75 degrees Celsius) at which time the cooling slows down, as represented by cooling transition portion 636A. Among other factors, the relatively low thermal mass of the heating first sheet 122 (e.g. a thin metal sheet, such as a foil) cools instantaneously after the initial power pulse ceases). Moreover, the relatively large thermal mass and substantially cooler temperature of the heat sink 430 enhances this rapid cooling in a moderate way without dominating the thermal behavior of the first sheet 122 and PCR mixture 107.

In one aspect, the cooling transition portion 636A in FIG. 9A, after the nearly instantaneous rapid cooling (from peak 615) corresponds to portion 709A in the power signal graph of FIG. 9B, during which no power (e.g. zero watts) is applied to the heating first sheet 122 of first wall 120 of the PCR well. This portion 709A may sometimes be referred to as no-heating portion or non-heating portion of the power signal profile 710 (FIG. 9B) and/or the temperature profile 610 (FIG. 9A). The portion 709A also may sometimes be referred to as being a power-neutral portion because no power is applied for a period of time.

The duration of the non-heating portion 709A of the power signal profile 710 is quite short as shown in FIG. 9B such as, but not limited to, about 0.25 seconds and is used to enhance the abrupt cooling of the first sheet 122 at least to a point 637 where the falling temperature is approaching the target second temperature (e.g. 60 degrees Celsius) of the second phase 632 of the temperature profile 610 in FIG. 9A.

At this point 637, the power signal (to heat the PCR mixture 107) is re-activated as represented by portion 711A in FIG. 9B, with the magnitude of the power signal being applied continuously and rising abruptly to establish and maintain the temperature at the second target temperature (e.g. 60 degrees), such as for annealing. The power applied in segment 711A rises abruptly in order to counteract the relatively larger thermal mass and substantially cooler temperature of the heat sink, as previously described in association with at least FIG. 6.

The abrupt heating portion 711A in FIG. 9B generally corresponds to, and generally coincides with, the segment 633 of second phase 632 (e.g. annealing) through which the PCR temperature is maintained at the second target temperature (e.g. 60 degrees Celsius, in some examples), such as for annealing.

Via this arrangement, the rapidly falling temperature of the PCR mixture 107 is abruptly prevented from falling any further by the local application of heat via the first sheet 122 of the first wall 120 to establish and maintain a second target temperature (e.g. an annealing temperature) for about a first portion of the thermal cycle 612.

As further shown in FIG. 9B, following portion 711A, the power signal profile 710 rises nearly instantaneously in portion 715 in order to abruptly raise the temperature of the first sheet 122 from the second target temperature (e.g. 60 degrees Celsius) to a third target temperature (e.g. 70 degrees Celsius), such as for elongation, as represented by the transition portion 636B of the PCR temperature profile in FIG. 9A.

With further reference to FIG. 9A, once the third target temperature (e.g. 70 degrees) is achieved for third phase 632, it is maintained for nearly 2 seconds, as represented by the linear segment 634 in FIG. 9A, by substantially reducing (e.g. exponentially) the power signal, as represented by portion 719A in FIG. 9B (but without terminating the power signal). By the time a total of 4 seconds (or other intended duration) has expired, the magnitude of the power signal has generally leveled off, such as at 20 Watts as shown in FIG. 9B, to maintain the third target temperature. At this point, the continuous power signal is deactivated such that the magnitude of the “continuous” power signal becomes zero Watts, as represented by segment 721 in FIG. 9B. This segment 721 generally corresponds to the transition portion 636C in FIG. 9A at the conclusion of the elongation segment 634 of the third phase 632. The segment 721 in FIG. 9B occurs just prior to, or nearly simultaneously with, the start of the next thermal cycle 612B (FIG. 9A) at which an initial rapid pulse of first phase 614 occurs, such as previously described for thermal cycle 612A in FIG. 9A.

It will be understood that a thermal cycle 612A, 612B, etc. having a duration of about 4 seconds is merely an example, and that in some examples, a thermal cycle 612A, 612B, and so on, may have a different duration which can be less than 4 seconds, such as at least about 2 seconds (e.g. 1.8, 1.9, 2, 2.1, 2.2), at least about 2.5 second (e.g. 2.3, 2.4, 2.5, 2.6, 2.7), at least about 3 seconds (e.g. 2.8, 2.9, 3, 3.1, 3.2), at least about 3.5 seconds (e.g. 3.3, 3.4, 3.5, 3.6, 3.7), or which can be greater than 4 seconds, such as at least about 4.5 second (e.g. 4.3, 4.4, 4.5, 4.6, 4.7), at least about 5 seconds, at least about 5.5 seconds (e.g. 5.3, 5.4, 5.5, 5.6, 5.7), at least about 6 seconds (e.g. 5.8, 5.9, 6, 6.1, 6.2), and so on.

These thermal cycles 612A, 612B, and so on, are repeated successively for a period of time to cause the PCR reaction process to occur for a sufficient number of thermal cycles to generate a sufficient number of amplicons, which in turn may increase a limit of detection, which in turn may increase an accuracy and robustness of the PCR test, such as via reducing false negatives regarding an analyte of interest, as previously described.

In some examples, various parameters associated with the example temperature profile of FIG. 9A (e.g. individual thermal cycles) may be selectable such as, but not limited to, a selection of the relative proportion of (e.g. 612A, 612B) as time spent in the second phase 632 (e.g. at the second target temperature), which may be represented by duration D3, and time spent in the third phase 634 (e.g. at the third target temperature), which may be represented by duration D3. In some such examples, about 50 percent (e.g. 49.5, 49.6, 49.7, 49.8, 49.9. 50, 50.1, 50.2, 50.3, 50.4) of a total duration (D3 plus D4) of the thermal cycle is spent in each of the respective second and third phases 632, 634. However, in some examples, about 45 percent (e.g. 44.5, 44.6, 44.7, 44.8, 44.9. 45, 45.1, 45.2, 45.3, 45.4, 45.5) of the total duration (D3 plus D4) may be spent in a respective one of the second and third phases 632, 634 and about 55 percent (e.g. 54.5, 54.6, 54.7, 54.8, 54.9. 55, 55.1, 55.2, 55.3, 55.4, 55.5) may be spend in the other respective second or third phase 632, 634. In some examples, about 40 percent (e.g. 39.5, 39.6, 39.7, 39.8, 39.9. 40, 40.1, 40.2, 40.3, 40.4, 40.5) of the total duration (D3 plus D4) may be spent in a respective one of the second and third phases and about 60 percent (e.g. 59.5, 59.6, 59.7, 59.8, 59.9. 60, 60.1, 60.2, 60.3, 60.4, 60.5) may be spent in the other respective second or third phase 632, 634. In some examples, other different selectable proportions may be implemented.

In some examples, at least one parameter associated with the example temperature profile of FIG. 9A may be selectable such as, but not limited to, the particular target second temperature and/or target third temperature. In the example shown in FIG. 9A, the target second temperature is selected as about 60 degrees Celsius (e.g. 59.5, 59.6, 59.7, 59.8, 59.9. 60, 60.1, 60.2, 60.3, 60.4, 60.5) while the target third temperature is selected as 70 degrees Celsius (69.5, 69.6, 69.7, 69.8, 69.9. 70, 70.1, 70.2, 70.3, 70.4, 70.5). However, in some examples, the target second temperature may be selected to be a single temperature, which falls between about 50 degrees Celsius (e.g. 49.5, 49.6, 49.7, 49.8, 49.9. 50, 50.1, 50.2, 50.3, 50.4) and about 65 degrees Celsius (e.g. 64.5, 64.6, 64.7, 64.8, 64.9. 65, 65.1, 65.2, 65.3, 65.4, 65.5). In some examples, the target second temperature may be selected to be a single temperature, which fall between about 55 degrees Celsius (e.g. 54.5, 54.6, 54.7, 54.8, 54.9. 55, 55.1, 55.2, 55.3, 55.4, 55.5) and about 65 degrees Celsius. In some examples, the target second temperature may be selected to be a single temperature, which falls between about 58 degrees Celsius (e.g. 57.5, 57.6, 57.7, 57.8, 57.9. 58, 58.1, 58.2, 58.3, 58.4, 58.5) and about 62 degrees Celsius (e.g. 61.5, 61.6, 61.7, 61.8, 61.9. 62, 62.1, 62.2, 62.3, 62.4, 62.5).

In some examples, at least one parameter associated with the example temperature profile of FIG. 9A may be selectable such as, but not limited to, the particular shape of the transition portions (e.g. 636A, 636B, 636C or 639A) and/or overall shape of the second and third phase portions 632, 634. For instance, the overall shape of the second and third phase portions 632, 634 may be selected to be as shown in FIG. 9A. However, in some other examples, the overall shape of the second and third phase portions 632, 634 may be selected and implemented to comprise a sinusoidal shape or other cyclical shape. In some examples, a shape of the transition portions (e.g. 636B, 636C) may depend, at least partially on, the magnitude of the difference between the second and third target temperatures and/or a selectable speed with which the transition is to be implemented.

In some examples, a response time for the first sheet 122 to cool (from the peak 615 of the first phase 614) to the second target temperature may be least partially based on a target temperature at which the heat sink (e.g. 430 in FIGS. 5-6) is maintained. In the example shown in FIG. 9A in which the target heat sink temperature is 35 degrees Celsius, it takes about 0.5 seconds to reach a target temperature (in second phase 632) of about 60 degrees Celsius and just 150 milliseconds to reach about 65 degrees Celsius on the way to the second target temperature of about 60 degrees Celsius. In the other example shown in FIG. 9A in which the target heat sink temperature is about 15 degrees Celsius, the response time to cool (from the peak temperature 615 of first phase 614) is a about 250 milliseconds to reach the target second temperature (of second phase 632) of the first sheet 122. Accordingly, while other heat sink temperatures may be selected and implemented, operation at these two different heat sink temperatures (e.g. about 15 degrees Celsius, 35 degrees Celsius) demonstrates that it may take about 50 percent longer to reach one example second target temperature (e.g. about 60 degrees Celsius) when the heat sink temperature is at a relatively higher temperature, such as 35 degrees Celsius, then when the heat sink temperature is at a relatively lower temperature, such as about 15 degrees Celsius.

As can be readily appreciated from the temperature profile 610 in FIG. 9A and the power signal profile 710 in FIG. 9B, by selectively electrically activating via the first sheet 122 of first wall 120 of PCR well 105 with varying amounts of power in a strategic manner, selectable temperatures of the first sheet 122 may be achieved and maintained for desired time intervals so as to implement precise and accurate heating of the portion of PCR mixture within the thermal cycling zone (Z). Via such arrangements, an efficiency and effectiveness of a PCR test may be significantly increased at least because each of the second and third phases can be performed for a relatively longer portion of a duration of a thermal cycle, rather than a longer period of time being consumed by a temperature of the first sheet 122 being in transition between first, second, and third target temperatures of the respective first, second, and third phases.

Moreover, by modulating the temperature of the PCR mixture 107 (in PCR well 105) predominantly by local heating with selective electrical activation of first sheet 122 in first wall 120, these example arrangements generally avoid hindrances that might otherwise be caused by thermal gaps between PCR mixture and a heat sink, due to types of materials and/or imperfections in construction and/or materials in the first wall 120 of the PCR well 105.

With further reference to FIGS. 9A and 9B, additional aspects of the temperature profile 610 (FIG. 9A) and/or the power signal profile 710 (FIG. 9B) are described for an example in which the heat sink temperature is maintained at 35 degrees Celsius, as represented by the below-noted dashed line portions in FIG. 9A and FIG. 9B.

With this in mind, as shown in FIG. 9A, a cooling transition portion 638A occurs, just after the nearly instantaneous rapid cooling (from peak 615) and corresponds to portion 709B in the power signal graph of FIG. 9B. In portion 709B, no power (e.g. zero watts) is applied to the heating first sheet 122 of first wall 120 of the PCR well. This portion 709B may sometimes be referred to as non-heating portion, a no-heating portion or a power-neutral portion, similar to portion 709A.

The duration of the non-heating portion 709B of the power signal profile 710 is slightly longer than non-heating portion 709A (for 15 C heat sink temperature) and may comprise about 0.4 or 0.5 seconds. Like non-heating portion 709A, the non-heating portion 709B may be used to enhance the abrupt cooling of the PCR mixture 107 at least to a point 637 (FIG. 9A) where the falling temperature is approaching the target second temperature (e.g. 60 degrees Celsius) of the annealing portion 632 of the temperature profile in FIG. 9A. It will be understood that point 637 of the temperature profile 610 for a heat sink temperature of 35 C may occur slightly later than when the heat sink temperature is about 15 C, in some examples.

At this point 637, the power signal (to heat the PCR mixture 107) is re-activated as represented by the starting point of portion 711B in FIG. 9B, with the magnitude of the power signal being applied continuously and rising abruptly to establish and maintain the temperature at the second target temperature (e.g. 60 degrees), such as for annealing. Like portion 711A, the power applied in portion 711B rises abruptly in order to counteract the relatively larger thermal mass and substantially cooler temperature of the heat sink, as previously described in association with at least FIG. 6.

The abruptly increasing heating portion 711B in FIG. 9B generally corresponds to, and generally coincides with, the segment 633 of the second phase 632 through which the first sheet 122 is maintained at the second target temperature (e.g. 60 degrees Celsius, in some examples), such as for annealing.

Via this arrangement, the rapidly falling temperature of the first sheet 122 is abruptly arrested by the local application of heat via the first sheet 122 of the first wall 120 to establish and maintain a second target temperature (e.g. an annealing temperature) for about of the thermal cycle 612.

As further shown in FIG. 9B, following portion 711B, the power signal rises nearly instantaneously in portion 715 in order to abruptly raise the temperature of the PCR mixture 107 from the second target temperature (e.g. 60 degrees Celsius) to a third target temperature (e.g. 70 degrees Celsius), such as for elongation, as represented by the transition portion 636B of the PCR temperature profile in FIG. 9A.

With further reference to FIG. 9A, once the third target temperature (e.g. 70 degrees) is achieved for third phase 632, it is maintained for nearly 2 seconds, as represented by the linear segment 634 in FIG. 9A, by substantially reducing (e.g. exponentially) the power signal, as represented by portion 719B in FIG. 9B (but without terminating the power signal). By the time a total of 4 seconds (or other intended duration) has expired, the magnitude of the power signal has generally leveled off, such as at 20 Watts as shown in FIG. 9B, to maintain the third target temperature. At this point, the continuous power signal is deactivated such that the magnitude of the “continuous” power signal becomes zero Watts, as represented by segment 721 in FIG. 9B. This segment 721 generally corresponds to the transition portion 636C in FIG. 9A at the conclusion of the elongation segment 634 of the third phase 632. The segment 721 in FIG. 9B occurs just prior to, or nearly simultaneously with, the start of the next thermal cycle 612B (FIG. 9A) at which an initial rapid pulse of first phase 614 occurs, such as previously described for thermal cycle 612A in FIG. 9A.

It will be further understood that this increased temperature control provided via examples of the present disclosure is further demonstrated by comparison of the temperature profile 610 (FIG. 9A) with a temperature profile 640, as represented by the dash-dot line(s) in FIG. 9A, for which the heat sink temperature is about 65 degrees Celsius.

In particular, the temperature profile 640 in FIG. 9A represents a temperature of a first sheet 122 (and therefore a portion of the PCR mixture 107 within the thermal cycling zone Z) during different phases of thermal cycling for an arrangement in which a heat sink temp is 65 degrees Celsius. In such arrangements, the heat sink temperature would have a value between, or close to one of, a second target temperature of the second phase 632 and a third target temperature of the third phase 643. This arrangement would stand in sharp contrast to examples of the present disclosure, such as examples in which a heat sink temperature is substantially less than a temperature of a second phase and/or third phase of a thermal cycle.

As shown in FIG. 9A, for each cycle 612A, 612B and so on, the temperature profile 640 in FIG. 9A comprises a second phase 642 (e.g. annealing) and a third phase 644 (e.g. elongation). As seen in FIG. 9A, immediately following the initial, rapid pulse of the first phase 614 (shown in solid lines) and immediate falling temperature of the first sheet 122, the second phase 642 slowly decreases from about 67 degrees to about 64 degrees until a transition portion 646B is reached at which the temperature rises just one or two degrees to about 66 degrees, at which point the third phase 644 starts. During the third phase 644, the temperature slightly rises from about 66 degrees to about 67 degrees, before starting to decrease again via transition 646C just before the initial rapid pulse of the first phase 614 of the next thermal cycle 612B. As apparent in viewing the temperature profile 640 in FIG. 9A, with the heat sink temperature at 65 degrees and the heat sink (e.g. 430) having a thermal mass substantially greater than a thermal mass of the heating first sheet 122, the temperature of the first sheet 122 (and therefore the portion of the PCR mixture 107 within the thermal cycling zone Z) is unable to cool to a desired target temperature (e.g. 60 degrees in some examples) for a second phase of a thermal cycle (of a three-phase PCR test) within a target time frame (e.g. 2 seconds). Moreover, in temperature profile 640 of FIG. 9A, the temperature of the first sheet 122 is unable to rise to a desired target temperature (e.g. 70 degrees) within a target time frame (e.g. 2 seconds) for a third phase of a thermal cycle.

In one aspect, in such arrangements, it is apparent that the thermal mass of the heat sink (e.g. 430 or other) and relatively high temperature of the heat sink (e.g. 430) dominate the thermal mass and selected temperature of the heating first sheet 122.

In such an arrangement like one in which the heat sink temperature is 65 degrees Celsius, the inability to reach two different target temperatures in the second and third phases would inhibit performance of the intention to perform annealing and elongation aspects of the PCR reaction process separately from each other in a sequential manner, thereby resulting an inadequate test within a comparable time frame and/or a test taking a significantly longer duration to complete.

Accordingly, by comparison with the relatively lethargic temperature profile 640 for the relatively warm heat sink temperature (e.g. 65 degrees Celsius), the temperature profile 610 for the heat sink temperatures of 15C and 35C, respectively, exhibit great agility in rapidly achieving a target temperature and/or transitioning between different target temperatures, such that at least some examples of the present disclosure are demonstrably fast so as to provide for two distinct target temperatures for two distinct phases of a thermal cycle within a relatively short duration, e.g. about 4 seconds or even less in some examples as previously described.

In some examples, the precise and agile manner of controlling the temperature of a portion of the PCR mixture 107 (within the thermal cycling zone Z), via control of the temperature of the heating first sheet 122 of first wall 120, may be enhanced via controlling a temperature of the heat sink (e.g. 430 in FIGS. 5-6) at a selected temperature which is substantially less than a temperature of the first sheet 122 (e.g. a temperature of the second phase, in some examples). With this in mind, FIG. 9C is a graph schematically representing the amount of cooling caused by (e.g. heat evacuated by) a heat sink (e.g. 430), for two different target heat sink temperatures, such as the example heat sink temperatures (e.g. 15 degrees Celsius and 35 degrees Celsius) for which a temperature profile 610 of a first sheet 122 was depicted in FIG. 9A. This cooling power may depend on the dimensions of the heat sink 430, PCR well 105, type of material from which the heat sink is formed and/or ambient temperature.

As shown as solid black lines in FIG. 9C, a cooling power profile 760 represents power in watts (as represented along a Y-axis 754) as plotted over time in seconds (as represented along an X-axis 753) shown in solid black lines). The cooling power profile 760 corresponds to the cooling power (e.g. heat evacuated) associated with, and/or of, a heat sink 430 as shown in FIGS. 5-6. As shown in FIG. 9C, more cooling power (e.g. 17 watts) is to occur during the third phase portions 762 (e.g. elongation) than during the second phase portions 763 (e.g. annealing). As shown in FIG. 9C, each power cycle 761A comprises a decreasing cooling power portion 764, a first constant cooling power portion 763, an increasing cooling power portion 765, and a second constant cooling power portion 762, with cycles 761A being repeating successively as long as thermal cycles of PCR mixture 107 (in FIG. 9A) are being performed in order to maintain the heat sink at a constant temperature of 15 degrees Celsius during such repeated thermal cycling of the PCR mixture 107 in PCR well 105. In some such examples, the first constant cooling power portion 763 generally corresponds to the second target temperature of the second phase (e.g. 632 in FIG. 9A), at which time the amount of cooling power is to be constant (e.g. 14.5 Watts, in some examples) to enable maintaining a constant second target temperature of the first sheet 122 (FIG. 1A-6) until reaching the next transition 765, i.e. increasing cooling power portion 765. In some examples, the second constant cooling power portion 762 generally corresponds to the third target temperature of the third phase (e.g. 634 in FIG. 9A), at which time the amount of cooling power is constant (e.g. about 17.5 Watts, in some examples) at which time the amount of cooling power is constant to enable constant third target temperature, at least until reach next decreasing power portion 764.

As further shown in FIG. 9C, the cooling power profile 760 includes a brief irregularity, depicted at point 767, which is a transition between an end of constant cooling power portion 762 (for a third phase) and a beginning of a decreasing cooling power portion 764 (for an end of a first phase and beginning of a second phase). This point 767 generally corresponds to the point at which the initial rapid pulse of the first phase 614 (FIG. 9A) occurs.

Via this example arrangement, the cooling power profile 760 of FIG. 9C depicts an example method including cycles of varying an amplitude of cooling power implemented by a cooling element (e.g. 544, 550 in FIGS. 6-7) of the heat sink (e.g. 430 in FIGS. 5-6) in order to maintain the heat sink at a constant temperature of 15 degrees Celsius. By providing a thermal mass of the heat sink, which is substantially larger than a thermal mass of the first sheet 122, and maintaining the heat sink (e.g. 430) at a constant temperature, which is substantially less than the target temperature of the first sheet 122, this arrangement enables precise and rapid changes in the respective selectable target temperatures of the first sheet 122 during the three temperature phases of the thermal cycles to perform the PCR test, as shown in FIG. 9A.

As further shown in FIG. 9C, in some examples cooling power profile 780 provides a similar profile of cooling power implemented by a cooling element (and/or heating element) in order to maintain the heat sink at a constant temperature (e.g. 35 degrees Celsius), which is substantially less than the respective second and third target temperatures (of the PCR mixture 107 and first sheet 122) during second and third phases of repeated thermal cycles, as shown in FIG. 9A. As compared to cooling power profile 760, the cooling power profile 780 applies lower amplitudes of power, but otherwise exhibits the same general shape/pattern as cooling power profile 760. As shown in FIG. 9C, each cooling power cycle 781A comprises a decreasing cooling power portion 784, a first constant cooling power portion 783, an increasing cooling power portion 785, and a second constant cooling power portion 782, with cycles 781A, 781B repeating successively as long as thermal cycles of PCR mixture 107 (in FIG. 9A) are being performed in order to maintain the heat sink at a constant temperature of 35 degrees Celsius during such repeated thermal cycling of the PCR mixture 107 in PCR well 105.

In some examples, prior to initiating thermal cycling (e.g. pulse amplification) within zone Z as described in association with at least FIGS. 9A-9C, the heating element 542 of the heat sink 430 (FIG. 6) may be used to manage the selectable temperature of the heat sink 430, which in turn helps bring the temperature of the PCR mixture 107 closer to a starting temperature (e.g. about 65 to about 75 degrees) prior to an initial rapid pulse (e.g. portion 615 in FIG. 9A) of a first thermal cycle 612A.

Conversely, once the thermal cycling (e.g. 612A, 612B in FIG. 9A) has commenced, in some examples just the cooling element 544 of the heat sink 430 (FIG. 6) may be used to manage the selectable temperature of the heat sink 430, which in turn helps manage the temperature of the first sheet 122 (and therefore the PCR mixture 107 within the thermal cycling zone Z) to achieve the target temperatures of at least the second and third temperature phases, and transition between the respective first, second, and third phases.

In some further examples, the heating element 542 of the heat sink 430 also may be used at the same time as the cooling element 544 to manage the selectable temperature of the heat sink 430, which in turn helps manage the temperature of the first sheet 122 (and therefore the PCR mixture 107 within the thermal cycling zone Z) to achieve the target temperatures of at least the second and third temperature phases (e.g. FIG. 9A, 9C), and transition between the respective first, second, and third phases. In particular, this use of the heating element 542 helps to smooth any abruptness, overshoots, or undershoots of intended temperatures of the heat sink 430 which might otherwise result from operation of the cooling element 544 alone.

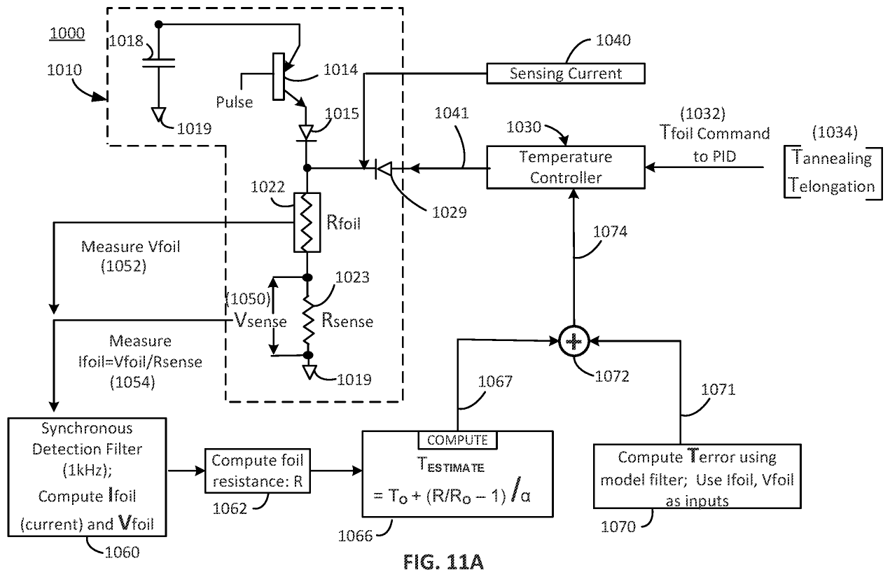

FIG. 10A is a diagram 800 including a sectional side view schematically representing an example testing device (and/or example testing method) including an example well 805 to receive a polymerase chain reaction (PCR) mixture 107. In some examples, the example well 805 may comprise at least some of substantially the same features and attributes as, and/or an example implementation of, the example wells (e.g. 105) as previously described in association with at least FIGS. 1A-9C. It will be understood that while some of the reference numerals previously shown in some of FIGS. 1A-9C are omitted for illustrative clarity/purposes, they are still generally applicable in FIGS. 10A-11C.

As further shown in FIG. 10A, in some examples the heating first sheet 122 may comprise a first portion 822A and a second portion 822B including segments 823, 824, which are on opposite sides of the first portion 822A. The first portion 822A of first sheet 122 is exposed to the PCR mixture 107 in the PCR well 105 generally, and in particular is exposed to and within the thermal cycling zone Z. Meanwhile, the second portion 822B of first sheet 122, including both segments 823, 824 is not exposed to the interior 109 of PCR well 105, such as the PCR mixture 107 within well 105.

It will be understood that in some examples, the first sheet 122 comprises a single element (e.g. foil) such that the designation of the first portion 822A and second portion 822B (and segments 823, 824) do not refer to separate elements but rather different regions of a monolithic (e.g. unitary) structure.

As shown in FIG. 10A, both the first portion 822A and the second portion 822B (including segments 823, 824) of the first sheet 122 are coupled relative to, and in thermal relation to, the second sheet 126 and the third sheet 128 which are vertically below first sheet 122. In some examples, when the third sheet 128 is releasably engaged relative to a heat sink (e.g. 430 in FIGS. 5-6), then the entire first sheet 122 is in thermal relation to (e.g. thermally coupled relative to) the heat sink.

Meanwhile, second walls 112, which define the interior 109 of the PCR well 105, are connected to the second portion 822B (including segments 823, 824) of the first sheet 122 and extend vertically above the second portion 822B of the first sheet 122. In contrast, the first portion 822A of the first sheet 122 is exposed to the interior 109 of PCR well 105 to be exposed to the PCR mixture 107, when present within the PCR well 105.

A cover 111 (one of the second walls 112) extends horizontally across the PCR well 105 and comprises outer portions 117A, 117B connected to, and in thermal relation to, the vertically extending second walls 112. An inner portion 117C of the cover 111 is interposed between the two outer portions 117A, 117B and in thermal relation to the interior 109 of the PCR well 105, which may be filled with the PCR mixture 107.

In some examples, the entire cover 111 (e.g. lid) is in thermal relation to the ambient environment 849 (e.g. air) external to the PCR well 105, which may exhibit a reference temperature Tₒ. One example reference temperature Tₒ may comprise about 20 degrees Celsius, which may correspond to one example ambient temperature external to the PCR well 105.

In some examples, the first portion 822A of first sheet 122 forms part of a first thermal vertical profile 832A (shown in as dash-dot-dot patterned line), while the second portion 822B (e.g. segments 823, 824) of the first sheet 122 forms part of a second thermal vertical profile 832B (shown in ordinary dashed lines 833, 834).

As further described in association with at least FIGS. 10B-11B, in some examples, an example method of determining a temperature of the PCR mixture 107 based measuring a resistance of the first sheet 122 (e.g. FIG. 1B) takes into account the first vertical thermal profile 832A (including the first portion 822A of the first sheet 122) differing from the second vertical thermal profile 832B. In particular, in doing so, the example method accurately differentiates between a temperature of the first portion 822A of the first sheet 122 (and therefore a temperature of the PCR mixture 107 within a thermal cycling zone Z) as compared to an average temperature of the first sheet 122, which comprises an average of a temperature of the first portion 822A and a temperature of the second portion 822B (e.g. segments 823, 824) of the first sheet 122. Because of a stronger cooling effect on first portion 822A caused by the entire volume of PCR mixture 107 within well 105, the first portion 822A of the first sheet 122 may exhibit a temperature which is lower than a temperature of the second portion 822B of the first sheet 122 because the second walls 112 (e.g. side walls) of the PCR well 105 vertically above the second portion 822B (e.g. segments 823, 824) retain more heat than the PCR mixture 107 vertically above the first portion 822A of the first sheet 122.

In some instance, this temperature difference before correction may comprise several degrees Celsius, which may be significant in at least some forms PCR testing where every effort is made to ensure accurate sensing of, and/or accurate implementation of, temperatures at which a given phase (e.g. denaturization, annealing, elongation) is intended to occur. If an actual temperature to be applied to the PCR mixture 107 which were significantly different than intended, then a greater or lesser amount of annealing may occur and/or a greater or lesser amount of elongation may occur than intended, which in turn may result in less amplicon being produced. This, in turn, may undesirably raise the limit of detection, delay amplification, and may lead to false negatives or other amplification error, thereby ultimately reducing an accuracy and/or reliability of the PCR testing.

With this in mind, it will be understood that a measured resistance of the first sheet 122 will generally correspond to an average temperature of the first sheet 122 (including both portions 822A and 822B) which does not account for the above-described heterogeneous arrangement of the first sheet 122 relative to other structures (e.g. second walls 112 and the interior 109).

Accordingly, at least some examples of the present disclosure may comprise applying a correction model to represent the first vertical thermal profile 832A (including the first portion 822A of the first sheet 122) being different from the second vertical thermal profile 832B (including the second portion 822B of the first sheet 122). In some examples, the correction model represents the thermal behavior and influence of the second portion 822B of the first sheet 122 (relative to the second portion 822B of the first sheet 822) being different from the thermal behavior and influence of the PCR mixture 107 relative to the first portion 822A of the first sheet 122.

In some such examples, the correction model may be expressed as representing a thermal behavior of at least one structure which further defines the well and which is in thermal relation with at least a second portion of the first sheet. For example, the at least one structure may comprise at least second walls 112 of the PCR well which extend vertically above the second portion 822B of the first sheet 122 and which are not present vertically above the first portion 822A of the first sheet 122.

With these structures and arrangements in mind, as shown in at least FIGS. 1A and/or 10A, in some examples, a device comprises a well 105 to receive a polymerase chain reaction (PCR) mixture 107 within an interior 109 partially defined by a first wall 110, which comprise a heating first sheet 122 and a control portion 130. The control portion 130 is to determine a first temperature of a first portion 822A (FIG. 10A) of the first sheet 122 exposed to the interior 109, based on monitoring a resistance of the first sheet 122 and based on a correction model, the correction model representing a thermal behavior of at least one structure which further defines the well 105 and which is in thermal relation with at least a second portion 822B of the first sheet 122. The control portion 130 (FIG. 1A) is also to, based on the determined first temperature, apply a first electrical signal to the heating first sheet 122 to heat the PCR mixture 107, at a selectable temperature, within a thermal cycling zone Z within the interior 109 in close thermal proximity to the first wall 110.

As further shown in FIG. 10A and as referenced further in association with at least FIGS. 101B-11C, one example temperature representative of a temperature of the first portion 822A of the first sheet 122 may correspond to a temperature at a region represented by dashed lines C.

With this general arrangement in mind, FIG. 10B is a graph 900 schematically representing a numeric simulation providing a comparison of example temperature profiles based on measuring a resistance of a heating element of an example well for receiving a polymerase chain reaction (PCR) mixture.

As shown in FIG. 10B, graph 900 represents a temperature (degrees Celsius) along Y-axis 904 relative to time (seconds) along an X-axis 902 for plotting a first temperature profile 910, a second temperature profile 940, and a third temperature profile 970. In some examples, the first temperature profile 910 represents a temperature of the first portion 822A of the first sheet 122 from a numeric simulation, with this temperature sometimes being referred to as well temperature or a temperature of the PCR mixture 107 within the well 105 such as the temperature of the PCR mixture within the thermal cycling zone Z (e.g. FIG. 10A or 1A). In some examples, this numerical simulation represented by curve 910 is based on a 3D simulation taking into account all relevant parameters including (but not limited to) heating by the first sheet 122, a thermal resistance of first wall 110, the thermal resistance of the PCR mixture 107 in the well 105, the thermal properties of second walls 112 or other structures which impact the first sheet 122 in all zones (e.g. vertical thermal profiles), the heat sink 430, an ambient temperature surrounding the PCR well 105, and/or other parameters. These numerical simulations of curve 910 are simulating, in this particular example, rapid and frequent changes in PCR well temperature that could be achieved using cooling power changes as shown in FIG. 10C.

As shown in FIG. 10B, in some examples the second temperature profile 940 comprises an estimated temperature of the entire first sheet 122 that would be represented by determining the temperature based on a measured resistance of the first sheet 122, in which the estimated temperature corresponds to an average temperature of the entire first sheet 122 including both the first portion 822A and the second portion 822B.

As shown in FIG. 10B, in some examples the third temperature profile 970 comprises an estimated temperature of the first portion 822A of the first sheet 122 that would be represented by the estimated temperature of profile 940 upon applying a correction model which accounts for the difference between the first vertical thermal profile 832A (including first portion 822A of first sheet 122) and the second vertical thermal profile 832B (including the second portion 822B of the first sheet 122).

In some examples, the correction model may depend on: (1) the thermal resistance and thermal capacitance of the materials used to make the well chip structure and heatsink shown in FIG. 5 or FIG. 10A; (2) a response time which sometimes could be derived from thermal resistance and thermal capacitance, as represented by the expression τ=R(thermal resistance)·C(thermal capacitance); (3) a temperature measured in the heat sink 430; (4) ambient temperature; (5) input power to the first sheet 122; (6) estimated temperature in the well 105; (7) and/or other parameters.

In general terms, each of the respective temperature profiles 910, 940, 970 illustrate a temperature in response to a test variable power input profile which mimics the type of variable power input which would be applied to the heating first sheet to produce a desired temperature profile of cycles of heating and cooling a portion of the PCR mixture 107 (within the thermal cycling zone Z) for performing a PCR test using PCR well 105. One example test variable power input profile 980 is shown in FIG. 10C.

As shown in FIG. 10C, the test variable power input profile 980 is plotted relative to an X-axis 982 of time (seconds) and a Y-axis 984 power applied to the first sheet 122 (e.g. heating foil) in Watts. The test variable power input profile 980 comprises a first portion 986 which represent an example pattern used to increase the temperature of the well 105, and portion 988 which represents an example pattern used to decrease the well 105 temperature as shown in FIG. 10B. These temperature changes shown in FIG. 10B simulate a fast change in temperature suitable to support profiles like the one shown in FIG. 9A which may be applied, in some examples, to perform a PCR test.

With this in mind, as further shown in FIG. 10B, in response to such a test power input profile (e.g. 980 in FIG. 10C), the first temperature profile 910 comprises a series of cycles 911A, 911B, 911C, 911D, 911E.

Each sample cycle 911A, 911B, 911C comprises a rapidly rising temperature first portion 912, a temperature peak 914, a decreasing temperature second portion 916, and a leveling temperature third portion 918. The sample cycles 911A, 911B, 911C may be produced via a test variable power input signal like portion 986 in FIG. 10C.

As further shown in FIG. 10B, the sample temperature cycles 911D, 911E may be produced via a power input signal in portion 988 of FIG. 10C where during the cooling time of the first sheet 122, no power is added. In this particular demonstration in FIG. 10B, each temperature cycle 911D, 911E may represent general cooling steps. In some examples, each temperature cycle 911D, 911E may comprise of a starting point 922, a moderately rising temperature first portion 924, and a slow rising temperature second portion 926. However, it should be noted that in other examples, the target temperature profile may look more like a step function with a flat temp, as shown in FIG. 9A or other profiles that generate effective PCR amplification.

As further shown in FIG. 10B, in response to the same test variable power input profile 980 of FIG. 10C, the first temperature profile 940 (shown as a dashed-dot line) of an estimated temperature of the entire first sheet 122 (when no temperature correction model is used) comprises a series of cycles 941A, 941B, 941C, 943A, 943B. Each sample cycle 941A, 941B, 941C comprises a rapidly rising temperature first portion 942, a temperature peak 944 and a steadily decreasing temperature second portion 946. These sample temperature cycles 941A, 941B, 941C demonstrate an error of a few degrees Celsius if used to estimate the temperature of PCR well 105 when input signal portion 986 (FIG. 10C) is applied to produce the cycles 911A, 911B, 911C of temperature profile 910 under circumstances in which no correction model is applied, i.e. just the average temperature of the entire first sheet 122 is used.

As further shown in FIG. 10B, the sample temperature cycles 943A, 943B may be produced via the same test variable power input signal portion 988 applied to produce cycles 911D, 911D of temperature profile 910. Each temperature cycle 943A, 943B comprises a starting point 952, a moderately rising temperature first portion 954, and a slow rising temperature second portion 956.

As further shown in FIG. 10B, the temperature profile 970 represents an estimated temperature of the first portion 822A of the first sheet 122 (and portion of PCR mixture 107 within the thermal cycling zone Z) upon applying the above-mentioned correction model (CM). As apparent from temperature profile 970 in FIG. 10B, the temperature profile 970 (e.g. short dashed lines) exhibits values which are substantially the same as the various portions (e.g. 912, 914, 916, 918) of cycles 911A-911C of temperature profile 910 except with portions 971A, 971B, 971C of the temperature profile 970 comprising values which differ slightly from the values of the portions 914, 916, 918 for cycles 911A, 9111B, 911C of temperature profile 910. Similarly, as further apparent from temperature profile 970 in FIG. 10B, the temperature profile 970 (e.g. short dashed lines) exhibits values which are substantially the same as the various portions (e.g. 922, 924, 926) of cycles 911D, 911E of temperature profile 910 except with portions 971D, 971E of the temperature profile 970 comprising values which differ slightly from the values of the portions 922, 924, 926 for cycles 911D, 911E of temperature profile 910.

Accordingly, among other observations, FIGS. 10B-10C schematically represent that via application of a correction model, examples of the present disclosure may accurately estimate a temperature of a first portion 822A of first sheet 122 and therefore a temperature of a portion of the PCR mixture 107 within PCR well 105, instead of attempting to rely on an estimated temperature (e.g. profile 940) which corresponds to an average temperature of the entire first sheet 122.

As can been seen from FIG. 10B, significant temperature differences may be observed between the estimated temperature profile 940 (e.g. average temperature of the entire first sheet 122) and the corrected estimate temperature profile 970 (e.g. temperature of first portion 822A of the first sheet 122).

Via this arrangement of accurately determining a temperature of the portion of the PCR mixture 107 within the thermal cycling zone Z (as a corrected estimated temperature of the first portion 822A of the first sheet 122), each phase of a PCR test may be implemented significantly more accurately and faster, which in turn, may result in a desired degree of amplification during each thermal cycle, thereby producing an acceptable total quantity of amplified genetic sample (e.g. amplicon). Reliably and quickly achieving a desired total quantity of the amplicon, in turn, may enhance a limit of detection for the particular analyte of interest, which may increase the accuracy and/or reliability of the particular PCR test, such as via decreasing the number of false negatives.

With this in mind, FIG. 11A is a diagram schematically representing an example device 1000 (and/or example method), including first circuitry portion 1010, for applying heat at selectable temperatures, which may be determined using a correction model, to perform a PCR test in a PCR well.

As shown in FIG. 11A, in some example implementations of the device 1000, a resistor element 1022 (also labeled as Rfoil) may correspond to (and/or represents) heating via first sheet 122 (e.g. heating foil) of the PCR well 105 (as previously described in association with at least FIGS. 1-10B), while a reference resistor 1023 (also labeled Rsense) in FIG. 11A corresponds to low Temperature Coefficient resistor placed in series with the resistance to be measured (Rfoil) to provide a reference value when sensing a resistance of the first sheet 122. This reference resistor 1023 is placed in series with the foil resistance (Rfoil) so that both resistors are subject to the same sensing current. In some examples, the voltage across both resistors 1022, 1023 is measured using a 4-wire measurement such that the voltage across the resistors 1022, 1023 are measured via sensing wires different than those used to provide the sensing and heating currents. The sensing wires (of the 4-wire measurement) are fed into a high impedance amplifier (hence no current being drawn) or directly into an Analog to digital converter (ADC).

As further shown in FIG. 11A, in some examples the previously-mentioned sensing current may be implemented via a sensing current input 1040 for applying a sensing current, via pathway 1041 and diode 1029, to at least the heating foil 1022 and resistance 1023 of the first sheet 122. In some examples, the sensing current input may comprise an AC signal of about 1 kilohertz.

As further shown in FIG. 11A, in some examples the example device 1000 may comprise a transistor 1014 for applying a rapid pulse signal (for a first phase of PCR test) to the heating foil (i.e. resistor 1022), with transistor 1014 using stored energy from a capacitor 1018 coupled to ground 1019. The transistor 1014 is electrically coupled to the heating foil 1022 via a diode 1015.

As further shown in FIG. 11A, in some examples the example device 1000 may comprise a temperature controller 1030 electrically coupled to the resistor 1022 (e.g. first sheet 122 or heating foil) via a diode 1029. The temperature controller 1030 implements commands (1032) to achieve, via heating by resistor 1022, a selectable temperature 1034 for annealing and/or elongation. In some examples, such selectable temperatures 1034 may be implemented as a continuous power mode of a power signal such as, but not limited to, the arrangement described in association with at least FIGS. 9A-9C.

In some such examples, the temperature controller 1030 may comprise a proportional-integral-derivative (PID) controller, which may employ feedback to provide continuously modulated control of a signal such as a power signal to the heating first sheet 1022 (e.g. 122 in FIGS. 1A-6). In some such examples, at least some of the feedback used as an input may comprise the input signal 1074 corresponding to a sensed temperature of the PCR mixture 107 (within the thermal cycling zone Z), which was determined via an estimated temperature 1067 based on a measured resistance of the first sheet 122 and a value 1071 produced from application of a correction model 1070, as further described below.

Upon the application of the sensing current (per input 1040), a voltage 1050 (also labeled Vsense) may be measured across the heating foil 1022 (corresponding to the first sheet 122) to measure a voltage 1052 (also labeled Vfoil) and a measured current 1054 (also labeled as Ifoil) may be obtained using Vfoil (1050) and Rsense (1023) parameters, as further represented at 1060 at which a synchronous filter is used to compute the current Ifoil and the voltage Vfoil. Using this information, a resistance of the foil (R) may be computed at 1062. This computed foil resistance R is fed into a computation at 1066, involving a reference resistance (Rₒ) of the foil at a reference temperature Tₒ (e.g. an ambient temperature, such as 20 degrees Celsius), in order to determine an estimated temperature (T_{ESTIMATE}) of the heating foil 1022 (e.g. entire first sheet 122 in PCR well 105). In some examples, this determination is performed via the equation:

T_{ESTIMATE}=Tₒ+(R/Rₒ−1)/α+Terror,
wherein α represents the temperature coefficient of resistivity (TCR). In some examples, the temperature coefficient of resistivity may sometimes be referred to as a resistance-change factor per degree Celsius of temperature change. Because the estimate based on the temperature coefficient resistivity of the material would only yield an average temperature different from the specific temperature of the PCR well 105 (e.g. temperature of the first portion of the first sheet) that is sought to be measured, a temperature sensing correction factor was introduced. The temperature sensing correction factor may sometimes be referred to as Terror. In some examples, the temperature sensing correction factor (e.g. value) Terror may be based on at least one of a geometry, a material, and a resistance sensitivity of at least the first sheet 122 (FIGS. 1A-6). The geometry of the first sheet 122 may comprise being generally rectangular (e.g. having a length and width), being circular (e.g. having a diameter), having a thickness, and/or other geometrical parameter. In some examples, the above-mentioned geometry (on which Terror may be at least partially determined) may further comprise a geometry of the at least one structure (in addition to the first sheet 122) further defining the well 105 such as, but not limited to, the structures, elements, relationships, etc. as described in association with at least FIG. 10A. In some such examples, at least one structure further defining the well 105 comprises second walls (e.g. 112 in FIG. 1A-6) which define side walls of the interior 109 of the PCR well 105 which contains the PCR mixture 107.

In some examples, the temperature coefficient of resistivity a and the correction model filter parameters are fixed for a particular configuration type of a PCR well and may be determined at a factory after manufacture or in the field just before or after use of the PCR well 105, whether for each test or just some tests. In some examples, this temperature coefficient of resistivity a (material property) may be determined for one or some sample PCR wells of a batch of PCR wells.

As further shown in FIG. 11A, in some examples the computed estimated temperature (T_{ESTIMATE}) is fed into a computation point 1072 at which a value 1071 produced by the correction model (CM) 1070 also is applied. In some examples, as shown at 1070 in FIG. 11A, the computed value 1071 from the correction model 1070 may be represented as T_{ERROR} and may be computed using a model filter with a foil current (I_{FOIL}) and a foil voltage (V_{FOIL}) as inputs. One example implementation of the computation of the value 1071 (e.g. T_{ERROR}) produced from the temperature correction model CM 1070 is described in association with at least FIG. 11B.

Upon adjusting, at computation point 1072, the computed estimated temperature T_{ESTIMATE} via the value 1071 (e.g. temperature correction value labeled as T_{ERROR}) produced from the temperature correction model (CF) 1070, a closed loop feedback path 1074 is provided to the temperature controller 1030 to enable the temperature controller 1030 to adjust the applied power signal (to the foil 1022) in order to implement and achieve a desired temperature of the first portion 822A of the first sheet 122 (and therefore a desired temperature of the portion of the PCR mixture 107 in the thermal cycling zone Z in the PCR well 105).

FIG. 11B is a diagram schematically representing an example device 1100 (and/or example method) for determining a correction model for an estimated temperature of a heating element of a PCR well. In some examples, the example device 1100 may comprise at least some of substantially the same features and attributes of the temperature correction model 1070 as previously described in association with at least FIG. 11A and more generally with at least FIGS. 10A-11A.

As shown in FIG. 11B, in some examples the example device 1100 may utilize and/or comprise a model filter 1110 by which an output of temperature error T_{ERROR} 1170 may be determined using heating power Q_{HEAT} 1120 and cooling power Q_{COOLING} 1130 as inputs. The model filter 1110 may sometimes be referred to as a digital filter 1110.

As shown in FIG. 11B, in some examples the heating power input 1120 may comprise, or be expressed as, power in watts according to a voltage (V_{FOIL}) of the heating foil (e.g. first sheet 122) multiplied times a current (I_{FOIL}) of the heating foil (e.g. first sheet 122). As further shown in FIG. 11B, in some examples the cooling power input 1130 may comprise, or be expressed as, a difference between a temperature of the well (e.g. portion of the PCR mixture 107 in the thermal cycling zone Z in the well 105) which could be taken as the first sheet 122 in the area 822A and a temperature of the heat sink (e.g. 430 in FIG. 6-7B), with the difference divided by a thermal resistance.

In one example, the Terror calculation shown in FIG. 11B, is based on the heating power input 1120 to the first sheet 122, the cooling power input 1130 affecting the first sheet 122, and several system thermal parameters which could be derived numerically, analytically, or experimentally. In some examples, the heating power input 1120 to the first sheet 122 is derived by its current and resistance (P=I\hat{ }2*R) and the cooling power input 1130 of the first sheet 122 is derived by the thermal difference of Twell as represented by first sheet 122, the heat sink temperature (T_{HS}) as measured directly, and the thermal resistance in between these two. In some examples, the system thermal parameters may comprise a thermal resistance of different materials, their thermal capacitance, and their response time, such as τ=R(thermal resistance)·C(thermal capacitance). In some examples, the cooling power input 1130 of the first sheet 122 may take into account the ambient temperature.

In some examples, in general terms the model filter 1110 corresponds to a mathematical model which represents a physical phenomenon by which the heating power input 1120 and the cooling power input 1130 are effectively filtered by a thermal mass of the structures of the PCR well 105, which together define an overall time constant. In some instances, a time period in which this thermal mass responds to heating power inputs 1120 and/or cooling power inputs 1130 may sometimes be referred to a thermal inertia of the structures of the PCR well 105 with regard to how the overall thermal mass, and thermal mass of different portions of the PCR well, responds to different temperature changes.

In one aspect, an overall time constant (of the thermal behavior of the structures of the PCR well 105) includes a first time constant associated with the local heating and cooling of the first sheet 122, which are intentionally used to heat and cool the PCR mixture 107 (e.g. the portion in the thermal cycling zone Z) in the PCR well 105. In some examples, the first time constant is relatively short and is on the order of hundreds of milliseconds, such as the approximately 100 milliseconds during which the first sheet 122 applies a rapid pulse portion (e.g. 614 in FIG. 9A) during a first phase of a PCR test and/or such as the approximately 500 milliseconds (in some examples) during which the first sheet 122 may cool (for a given heat sink temperature such as 40 degrees Celsius) from the peak (e.g. 615 in FIG. 9A) of the rapid pulse portion of the power signal profile.

In some examples, the overall time constant may comprise a second time constant which is relatively long and which corresponds to a time for the entire system (e.g. structures of the PCR well) to reach equilibrium at a fixed power input, such as about 50 to about 60 seconds with the second time constant being on the order of about 20 seconds.

In some examples, the overall time constant may comprise a third time constant which is relatively short and which is faster than the time frame of the first time constant, and which therefore has a negligible effect on the error (discrepancy) between the temperature of the first portion 822A of the first sheet 122 (e.g. temperature profile 910 in FIG. 10A) and the estimated average temperature (e.g. estimated temperature profile 940 in FIG. 10A) of the entire first sheet 122.

In some examples, the model filter 1110 (e.g. digital model filter) may comprise a linear filter, which models the thermal system (e.g. structures of the PCR well) as behaving according to a first order.

In some examples, the model filter 1110 (e.g. digital model filter) may comprise a high pass filter 1122 and a low pass filter 1124 relating to the heating power 1120 and a high pass filter 1132 and a low pass filter 1134 relating to the cooling power 1130. In some such examples, the high pass filter 1122 for the heating power 1120 and the high pass filter 1132 for the cooling power 1130 both act to digitally filter out temperature changes which happen slower than the overall system thermal response, such as shown in FIGS. 9A-10B, because those relatively slower temperature changes (e.g. on the order of 50 to 60 seconds, and not shown in FIGS. 9A-10B) are associated with the above-named second time constant. As previously mentioned these temperature changes (associated with the second time constant) have a negligible effect on an error (discrepancy) between the temperature of the first portion 822A of the first sheet 122 (e.g. temperature profile 910 in FIG. 10A) and the average temperature (e.g. estimated temperature profile 940 in FIG. 10A) of the entire first sheet 122.

In some such examples, the lower pass filter 1124 for the heating power 1120 and the high pass filter 1134 for the cooling power 1130 both act to digitally filter out local time constants for the heating power (e.g. heating by the first sheet 122) and the cooling power (e.g. cooling from the first sheet 122 to the heat sink 430) which happen generally faster than the thermal response (e.g. the thermal response shown in FIGS. 9A-10B) of the local heating and cooling at first sheet 122. In some examples, these faster temperature changes may correspond to the above-named third time constant with those relatively fast temperature changes (e.g. local time constants) have a negligible effect on an error (discrepancy) between the temperature of the first portion 822A of the first sheet 122 (e.g. temperature profile 910 in FIG. 10A) and the average temperature (e.g. estimated temperature profile 940 in FIG. 10A) of the entire first sheet 122.

Together, the high pass filters 1122, 1132 and the low pass filters 1124, 1134 for the respective heating power input 1120 and the cooling power input 1130 enable determination at 1140 of a temperature correction value (e.g. T_{ERROR}) 1171, which is produced from the correction model 1070 in FIG. 11A.

In some examples, the model filter 1110 may be implemented as part of the correction model 1170, which in turn may comprise part of a control portion, such as but not limited to, a microcontroller by which the filter 1110 may perform the digital filtering to compute T_{ERROR} 1171 (e.g. from model 1070 in FIG. 11A) in real time during a PCR test or time period related to performing a PCR test.

FIG. 11C is a diagram schematically representing an example method 1150, which may be used to implement temperature sensing via the correction model 1170 of FIG. 11A and the digital model filter 1110 of FIG. 11B. In some examples, the example method 1150 may comprise or utilize at least some of substantially the same features and attributes as the examples described in association with at least FIGS. 10A-11B.

As shown at 1152 in FIG. 11C, method 1150 may comprise performing: (1) a numerical simulation to determine a temperature (T_{WELL}) of the first portion of the first sheet 122 (which is exposed to an interior 109 of a PCR well 105), which corresponds to the temperature of the PCR mixture 107 within the thermal cycling zone Z; and (2) determining an estimated temperature using the computed value (T_{ERROR}) from a correction model. The results may be expressed in a manner similar to that shown in FIG. 10B.

As shown at 1154 in FIG. 11C, method 1150 may comprise comparing the numerically simulated (T_{WELL}) with the corrected estimated temperature. Based on this comparison, at 1156 in FIG. 11C the method 1150 may select or adjust filter parameters of a model filter (e.g. 1110 in FIG. 11B) in order to minimize a difference between the numerically simulated (T_{WELL}) and the corrected estimated temperature.

As shown at 1158 in FIG. 11C, method 1150 may comprise computing a correction value (T_{ERROR}) from the correction model using the model filter (e.g. 1110 in FIG. 11B) according to the selected and/or adjusted filter parameters (e.g. model filter parameters), such as overall thermal response of the PCR well, local time constants of components of the PCR well and system gains (e.g. output to input gain e.g. temperature change per unit power of heating or cooling).

At 1152 in FIG. 11C, using the computed correction value (according to the selected/adjusted filter parameters) the method 1150 then performs another iteration of performing a numerical simulation to determine (T_{WELL}) and to determine a corrected estimated temperature, as previously described. At 1154, the comparison of (T_{WELL}) and the corrected estimated temperature is performed again. If the difference between the two meets a selectable criteria (e.g. within a selectable threshold) as represented by a YES path 1170, then the method 1150 may terminate at 1160 with the conclusion of having determined the target filter parameters for the correction model (e.g. 1170 in FIG. 11A). In some examples, the selectable criteria may comprise a temperature difference such as about 1 (e.g. 0.98, 0.99, 1, 1.01, 1.02) degree Celsius, or in some other examples a temperature difference of about 0.5 (e.g. 0.48, 0.49, 0.5, 0.51, 0.52) degrees Celsius.

Conversely, if the difference between T_{WELL} and the corrected estimated temperature (T_{CORRECTED}) does not meet the selectable criteria (e.g. a selectable temperature difference) as represented via the NO path 1172, then the method 1150 may continue with adjusting the filter parameters at 1156, performing the computation of the correction value at 1158, and again running the numerical simulation, etc. at 1152.

This cycle may be repeated as many times as suitable until the comparison at 1154 produces a temperature difference that meets the selectable criteria.

In some examples, the various components, profiles, filters, etc. of the examples of the present disclosure in FIGS. 10A-11C may comprise at least some of substantially the same features and attributes as, an example implementation of, and/or at least partially be implemented via the operations engine 1200 of FIG. 12 and/or the control portion 1300 of FIGS. 13A-13B.

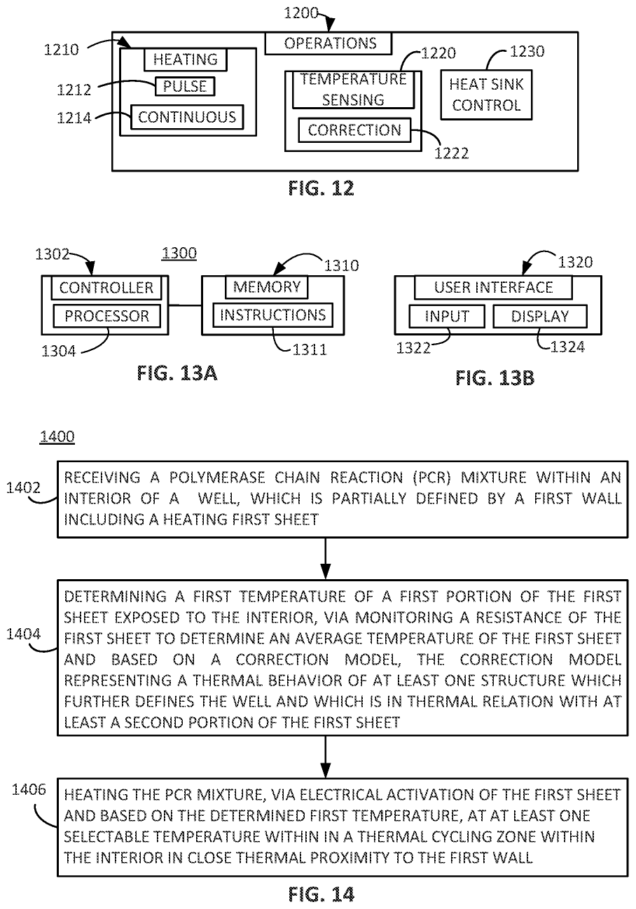

FIG. 12 is a block diagram schematically representing an example operations engine 1200. In some examples, the operations engine 1200 may form part of a control portion 1300, as later described in association with at least FIG. 13A, such as but not limited to comprising at least part of the instructions 1311. In some examples, the operations engine 1200 may be used to implement at least some of the various example devices and/or example methods of the present disclosure as previously described in association with FIGS. 1A-11 and/or as later described in association with FIGS. 13A-14. In some examples, the operations engine 1200 (FIG. 12) and/or control portion 1300 (FIG. 13A) may form part of, and/or be in communication with, a testing device (including at least one polymerase chain reaction (PCR) well) such as the example devices and methods described in association with at least FIGS. 1-11B and 13A-14.

In some examples and in general terms, the operations engine 1200 reports information of, monitors, and/or directs a polymerase chain reaction (PCR) test to occur within at least one well of a testing device. As shown in FIG. 12, in some examples the operations engine 1200 may comprise a heating engine 1210, a temperature sensing engine 1220, and/or a heat sink control engine 1230.

In some examples, the heating engine 1210 may track and/or control heating within a PCR well, such as via the three heating phases of a thermal cycle per temperature profile 610 in FIG. 9A, per power signal profile 710 in FIG. 9B, and/or per other example parameters throughout examples of the present disclosure. In some such examples, the heating engine 1210 may comprise a pulse parameter 1212 to track and/or control application of power to a heating element (e.g. first sheet 122) as an initial rapid pulse (e.g. 614 in FIG. 9A; 705 in FIG. 9B) in a first phase of a thermal cycle to provide heating at a first target temperature. In some such examples, the heating engine 1210 may comprise a continuous parameter 1214 to track and/or control application of power to a heating element (e.g. first sheet 122) in a continuous manner in a second phase (e.g. 632 in FIG. 9A) and a third phase (e.g. 634 in FIG. 9A) of a thermal cycle to provide heating at respective second and third target temperatures. In some such examples, the heating engine 1210 may cooperate with, and/or control, heating via the first sheet 122 in association with FIGS. 1A-9C and/or as otherwise described throughout examples of the present disclosure.

In some examples, the temperature sensing engine 1220 may track and/or control sensing a temperature of the first sheet 122 in order to help control a temperature of the first sheet 122 for heating, in three different phases, a portion of a PCR mixture within a thermal cycling zone (within a PCR well) in close thermal proximity the first sheet 122. In some such examples, the temperature sensing engine 1220 may cooperate with, and/or control, the temperature sensing elements in association with FIGS. 1B-1D and/or as described throughout examples of the present disclosure.

In some examples, the temperature sensing engine 1220 may comprise a correction model parameter 1222 to provide a more accurate measurement of a temperature of the portion of the PCR mixture 107 within the thermal cycling zone Z of the well (e.g. 105), which corresponds to a temperature of the first portion 822A of the first sheet 122 of the well, as shown in FIG. 10A. The correction model parameter 1222 accounts for the heterogeneous structure of the PCR well 105 in which the first portion 822A of the first sheet 122 is exposed to the interior 109 of the PCR well 105 (and its contents such as the PCR mixture 107) while the second portion 822B of the first sheet 122 is in direct thermal contact with the second walls 122 (instead of the interior 109 and/or PCR mixture 107). In some examples, the correction model parameter 1222 may be implemented according to the example methods (and/or example devices) of at least FIGS. 10A-11C.

In some examples, the heat sink control engine 1230 may track and/or control the maintaining of, and/or sensing of, a temperature of a heat sink (e.g. 430 in FIGS. 5-6) to enhance temperature control of the first sheet 122 for heating, in three different phases, a portion of a PCR mixture within a thermal cycling zone (within a PCR well) in close thermal proximity to the first sheet 122. In some such examples, the heat sink control engine 1230 may cooperate with, and/or control, the various elements of heat sink control system in association with FIGS. 6-7 and/or as otherwise described throughout examples of the present disclosure.

It will be understood that in some examples, the heating engine 1210, the temperature sensing engine 1220, and/or the heat sink control engine 1230 may operate interdependently with each other in a manner consistent with various examples throughout the present disclosure in which heating, temperature sensing, and/or cooling are used to implement different temperature phases (and transitions therebetween) of a PCR test in association with FIGS. 1A-15. It will be further understood that in some examples, the heat sink control engine 1230 may play a role in heating in addition to cooling.

FIG. 13A is a block diagram schematically representing an example control portion 1300. In some examples, control portion 1300 provides one example implementation of a control portion forming a part of, implementing, and/or generally managing the example testing devices (e.g. molecular testing devices), including the particular portions, components, PCR wells, signal sources, temperature sensing elements, temperature sensing correction models, digital model filters, heating elements, heat sinks, heat sink control systems, cooling elements, operations, control portion, instructions, engines, functions, parameters, and/or methods, as described throughout examples of the present disclosure in association with FIGS. 1-12 and 13B-14. In some examples, control portion 1300 includes a controller 1302 and a memory 1310. In general terms, controller 1302 of control portion 1300 comprises at least one processor 1304 and associated memories. The controller 1302 is electrically couplable to, and in communication with, memory 1310 to generate control signals to direct operation of at least some of the example molecular testing devices, including the particular portions, components, wells, signal sources, temperature sensing elements, temperature sensing correction models, digital model filters, heating elements, heat sinks, heat sink control systems, cooling elements, operations, control portion, instructions, engines, functions, parameters, and/or methods, as described throughout examples of the present disclosure. In some examples, these generated control signals include, but are not limited to, employing instructions 1311 stored in memory 1310 to at least direct and manage testing operations via examples of the present disclosure. In some instances, the controller 1302 or control portion 1300 may sometimes be referred to as being programmed to perform the above-identified actions, functions, etc.

In response to or based upon commands received via a user interface (e.g. user interface 1320 in FIG. 13B) and/or via machine readable instructions, controller 1302 generates control signals as described above in accordance with at least some of the examples of the present disclosure. In some examples, controller 1302 is embodied in a general purpose computing device while in some examples, controller 1302 is incorporated into or associated with at least some of the example molecular testing devices, as well as the particular portions, components, PCR wells, signal sources, temperature sensing elements, temperature sensing correction models, digital filters, heating elements, heat sinks, heat sink control systems, cooling elements, operations, control portion, instructions, engines, functions, parameters, and/or methods, etc. as described throughout examples of the present disclosure.

For purposes of this application, in reference to the controller 1302, the term “processor” shall mean a presently developed or future developed processor (or processing resources) that executes machine readable instructions contained in a memory or that includes circuitry to perform computations. In some examples, execution of the machine readable instructions, such as those provided via memory 1310 of control portion 1300 cause the processor to perform the above-identified actions, such as operating controller 1302 to implement testing operations via the various example implementations as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 1310. The machine readable instructions may include a sequence of instructions, a processor-executable machine learning model, or the like. In some examples, memory 1310 comprises a computer readable tangible medium providing non-volatile storage of the machine readable instructions executable by a process of controller 1302. In some examples, the computer readable tangible medium may sometimes be referred to as, and/or comprise at least a portion of, a computer program product. In other examples, hard wired circuitry may be used in place of or in combination with machine readable instructions to implement the functions described. For example, controller 1302 may be embodied as part of at least one application-specific integrated circuit (ASIC), at least one field-programmable gate array (FPGA), and/or the like. In at least some examples, the controller 1302 is not limited to any specific combination of hardware circuitry and machine readable instructions, nor limited to any particular source for the machine readable instructions executed by the controller 1302.

In some examples, control portion 1300 may be entirely implemented within or by a stand-alone device.

In some examples, the control portion 1300 may be partially implemented in one of the example testing devices and partially implemented in a computing resource separate from, and independent of, the example devices but in communication with the example testing devices. For instance, in some examples control portion 1300 may be implemented via a server accessible via the cloud and/or other network pathways. In some examples, the control portion 1300 may be distributed or apportioned among multiple devices or resources such as among a server, a testing device, a user interface.

In some examples, control portion 1300 includes, and/or is in communication with, a user interface 1320 as shown in FIG. 13B. In some examples, user interface 1320 comprises a user interface or other display that provides for the simultaneous display, activation, and/or operation of at least some of the example testing devices, including the particular portions, components, PCR wells, signal sources, temperature sensing elements, temperature sensing correction models, digital model filters, heating elements, heat sinks, heat sink control systems, cooling elements, operations, control portion, instructions, engines, functions, parameters, and/or methods, etc., as described in association with FIGS. 1-13A and 14. In some examples, at least some portions or aspects of the user interface 1320 are provided via a graphical user interface (GUI), and may comprise a display 1324 and input 1322.

FIG. 14 is a flow diagram of an example method 1500. In some examples, method 1500 may be performed via at least some of the example testing devices, including the particular portions, components, PCR wells, signal sources, temperature sensing elements, temperature sensing correction models, digital model filters, heating elements, heat sinks, heat sink control systems, cooling elements, operations, control portions, engines, functions, parameters, and/or methods, etc. as previously described in association with at least FIGS. 1-13B. In some examples, method 1500 may be performed via at least some testing devices, including the particular portions, components, PCR wells, signal sources, temperature sensing elements, temperature sensing correction models, digital model filters, heating elements, heat sinks, heat sink control systems, cooling elements, operations, control portions, engines, functions, parameters, and/or methods, etc. other than those previously described in association with at least FIGS. 1-13B.

As shown at 1402 in FIG. 14, in some examples method 1400 comprises receiving a polymerase chain reaction (PCR) mixture within an interior of at least one well, which is partially defined by a first wall including a heating first sheet. As further shown at 1404 in FIG. 14, in some examples method 1400 comprises determining a first temperature of a first portion of the first sheet exposed to the interior, via monitoring a resistance of the first sheet to determine an average temperature of the first sheet and based on a correction model, the correction model representing a thermal behavior of at least one structure which further defines the well and which is in thermal relation with at least a second portion of the first sheet. As further shown at 1406 in FIG. 14, in some examples method 1500 comprises heating the PCR mixture, via electrical activation of the first sheet and based on the determined first temperature, at at least one selectable temperature within in a thermal cycling zone within the interior in close thermal proximity to the first wall.

In some examples, method 1400 may further comprise determining of the first temperature of the first portion of the first sheet via generating, via the correction model, a temperature correction value to be applied to the average temperature of the first sheet, wherein the correction model at least partially represents the thermal behavior according to at least one of: a digitally filtered heating power of the first sheet during heating; and a digitally filtered cooling power associated with the at least one structure, which comprises a heat sink in indirect thermal contact with the first sheet.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. A device comprising:
a well to receive a polymerase chain reaction (PCR) mixture within an interior partially defined by a first wall, which comprises a heating first sheet; and
a control portion to:
determine a first temperature of a first portion of the first sheet exposed to the interior, based on monitoring a resistance of the first sheet and based on a correction model, the correction model representing a thermal behavior of at least one structure which further defines the well and which is in thermal relation with at least a second portion the first sheet; and
based on the determined first temperature, apply a first electrical signal to the heating first sheet to heat the PCR mixture, at a selectable temperature, within a thermal cycling zone within the interior in close thermal proximity to the first wall.

2. The device of claim 1, wherein the control portion is to determine the first temperature of the first sheet based on a temperature coefficient of resistivity of the first sheet and a linear relationship of the determined first temperature and the resistance.

3. The device of claim 1, wherein the monitored resistance of the first sheet corresponds to an average temperature of the first sheet.

4. The device of claim 3, wherein the control portion is to determine the first temperature of the first portion of the first sheet, by generating via the correction model, a temperature correction value to be applied to the average temperature of the first sheet, wherein the correction model at least partially represents the thermal behavior according to at least one of:
a heating power of the first sheet during heating; and
a cooling power associated with the at least one structure, which comprises a heat sink in indirect thermal contact with the first sheet.

5. The device of claim 4, wherein the correction model is to at least partially represent the thermal behavior by digitally filtering the heating power to remove at least one of:
a first temperature change occurring slower than an overall thermal response of the well; and
a second temperature change faster than local time constants of the first sheet.

6. The device of claim 4, wherein the correction model is to at least partially represent the thermal behavior by digitally filtering the cooling power to remove at least one of:
a third temperature change occurring slower than the overall thermal response of the well connected to a heat sink; and
a fourth temperature change faster than local time constants of the well.

7. The device of claim 1, wherein the thermal behavior represented by the correction model comprises a temperature coefficient of resistivity and model filter parameters, each of which are based on at least one of:
a geometry of at least the first sheet;
a type of material of the first sheet;
a sensitivity of the resistance of the first sheet to temperature changes; and
a geometry of the at least one structure further defining the well.

8. The device of claim 7, wherein the at least one structure further defining the well comprises at least second walls defining, in combination with the first wall, the interior of the well.

9. The device of claim 1, wherein the control portion is to cause, via the application of the first electrical signal, the thermal cycling zone to exhibit different heating phases operating in sequence at:
a first temperature comprising at least 90° C.;
a second temperature comprising at least about 25° C. less than the first temperature; and
a third temperature comprising at least about 5° C. greater than the second temperature and at least about 15° C. less than the first temperature.

10. The device of claim 9, wherein the control portion is to cause operation of the different heating phases as:
a first phase operating at the first temperature and comprising a first duration comprising no more than 10 milliseconds;
a second phase operating at the second temperature and comprising a duration of at least 300 milliseconds; and
a third phase operating at the third temperature and comprising a duration of at least 300 milliseconds.

11. A device comprising:
a well to receive a polymerase chain reaction (PCR) mixture within an interior partially defined by a first wall, which comprise a heating first sheet; and
a control portion to:
determine a first temperature of a first portion of the first sheet exposed to the interior, based on monitoring a resistance of the first sheet to determine an average temperature of the first sheet and based on a correction model, the correction model representing thermal behavior of at least one structure further defining the well and in thermal relation with at least a second portion of the first sheet, wherein the correction model is based on at least one model digital filter accounting for at least one of a geometry, a material, and a resistance sensitivity of at least the first sheet, and a geometry of the at least one structure further defining the well.

12. The device of claim 11, wherein the control portion is to determine the first temperature of the first portion of the first sheet, by generating via the correction model, a temperature correction value to be applied to the average temperature of the first sheet, wherein the correction model at least partially represents the thermal behavior according to at least one of:
a heating power of the first sheet during heating; and
a cooling power associated with the at least one structure, which comprises a heat sink in indirect thermal contact with the first sheet.

13. The device of claim 12, wherein the correction model is to at least partially represent the thermal behavior by digitally filtering:
the heating power to remove at least one of:
a first temperature change occurring slower than an overall thermal response of the well; and
a second temperature change faster than local time constants of the first sheet; and
the cooling power to remove at least one of:
a third temperature change occurring slower than the overall thermal response of the well connected to a heat sink; and
a fourth temperature change faster than local time constants of the well.

14. A method comprising:
receiving a polymerase chain reaction (PCR) mixture within an interior of a well, which is partially defined by a first wall including a heating first sheet;
determining a first temperature of a first portion of the first sheet exposed to the interior, via monitoring a resistance of the heating element to determine an average temperature of the first sheet and based on a correction model, the correction model representing a thermal behavior of at least one structure which further defines the well and which is in thermal relation with at least a second portion of the first sheet; and
heating the PCR mixture, via electrical activation of the first sheet and based on the determined first temperature, at at least one selectable temperature within in a thermal cycling zone within the interior in close thermal proximity to the first wall.

15. The method of claim 14 wherein the determining of the first temperature of the first portion of the first sheet comprises:
generating via the correction model, a temperature correction value to be applied to the average temperature of the first sheet, wherein the correction model at least partially represents the thermal behavior according to at least one of:
a digitally filtered heating power of the first sheet during heating; and
a digitally filtered cooling power associated with the at least one structure, which comprises a heat sink in indirect thermal contact with the first sheet.
